(19) United States
(12) Patent Application Publication
(10) Pub. No.: US 2023/0310865 A1
(43) Pub. Date: Oct. 5, 2023

(54) NON-CONTACT MONITORING FOR NIGHT TREMORS OR OTHER MEDICAL CONDITIONS

(71) Applicant: Covidien LP, Mansfield, MA (US)

(72) Inventors: Paul S. ADDISON, Edinburgh (GB); Adam J. RIVARD, Blaine, MN (US); Thomas I. MILLER, Blaine, MN (US); Sajith ANANTHARAMAN, Maple Grove, MN (US); Robert T. SANDGREN, Lindstrom, MN (US); Jeffrey P. BODNER, Plymouth, MN (US); Avram SCHEINER, Vadnais Heights, MN (US); Ryan B. SEFKOW, Plymouth, MN (US)

(21) Appl. No.: 18/295,717

(22) Filed: Apr. 4, 2023

Related U.S. Application Data
(60) Provisional application No. 63/327,218, filed on Apr. 4, 2022.

(51) Int. Cl. A61N 1/36; G16H 50/20; G16H 40/67; G16H 20/30; A61B 5/11

(52) U.S. Cl. CPC A61N 1/36139; G16H 50/20; G16H 40/67; G16H 20/30; A61B 5/1126

(57) ABSTRACT

A system may generate a waveform signal indicative of a biometric parameter of a target subject based on motion data associated with the target subject. The motion data may be provided by a depth sensing device and be substantially free of image data. The system may set a threshold condition based on one or more characteristics of the waveform signal. The system may output a control signal to a stimulation device based on the waveform signal satisfying the threshold condition.

### CROSS-REFERENCE TO RELATED APPLICATIONS

The present application claims benefit of priority to U.S. Provisional Patent Application No. 63/327,218, entitled “NON-CONTACT MONITORING FOR NIGHT TREMORS OR OTHER MEDICAL CONDITIONS” and filed on Apr. 4, 2022, which is specifically incorporated by reference herein for all that it discloses or teaches.

### FIELD OF INVENTION

The present disclosure is generally directed to medical monitoring, and relates more particularly to non-contact monitoring for patient conditions.

### BACKGROUND

Some systems may support non-contact monitoring and detection of patient conditions. In some cases, the systems may provide collected data to a medical provider for diagnostic and/or therapeutic purposes. Improved monitoring, detection, and treatment techniques are desired.

### BRIEF SUMMARY

Example aspects of the present disclosure include:

A system including: a processor; and a memory storing instructions thereon that, when executed by the processor, cause the processor to: generate a waveform signal indicative of a biometric parameter of a target subject based at least in part on motion data associated with the target subject; set a threshold condition based at least in part on one or more characteristics of the waveform signal; and output a control signal to a stimulation device based at least in part on the waveform signal satisfying the threshold condition.

Any of the aspects herein, wherein the instructions are further executable by the processor to: collect data associated with the target subject, wherein collecting the data includes: receiving, from a depth sensing device, the motion data associated with the target subject.

Any of the aspects herein, wherein collecting the data includes: receiving, from one or more sensing devices, one or more biometric measurements associated with the target subject, wherein generating the waveform signal is based at least in part on the one or more biometric measurements.

Any of the aspects herein, wherein the motion data is substantially free of image data.

Any of the aspects herein, wherein the instructions are further executable by the processor to: compare the one or more characteristics of the waveform signal to a target set of criteria, wherein setting the threshold condition includes maintaining or modifying the threshold condition based at least in part on a result of the comparison.

Any of the aspects herein, wherein the one or more characteristics of the waveform signal include a pattern of the waveform signal.

Any of the aspects herein, wherein the one or more characteristics of the waveform signal include a ratio associated with a first amplitude of the waveform signal and a second amplitude of the waveform signal.

Any of the aspects herein, wherein the instructions are further executable by the processor to: identify a quantity of apnea events, a quantity of hypopnea events, or both in association with a temporal duration, based at least in part on the waveform signal, wherein setting the threshold condition is based on the quantity of apnea events, the quantity of hypopnea events, or both in association with the temporal duration.

Any of the aspects herein, wherein the biometric parameter includes a respiration rate or a tidal volume.

Any of the aspects herein, wherein the instructions are further executable by the processor to: deliver, via the stimulation device, a therapy treatment to the patient based at least in part on the control signal.

Any of the aspects herein, wherein the instructions are further executable by the processor to: provide clinical data to a medical provider, wherein the clinical data includes a data record associated with the biometric parameter.

Any of the aspects herein, wherein the instructions are further executable by the processor to: output an alert based at least in part on the value of the biometric parameter, the one or more characteristics of the waveform signal, or both.

Any of the aspects herein, wherein the instructions are further executable by the processor to: calculate one or more values of the biometric parameter based at least in part on the motion data.

Any of the aspects herein, wherein the instructions are further executable by the processor to: provide at least a portion of the motion data to a machine learning model; receive an output from the machine learning model in response to the machine learning model processing at least the portion of the motion data, the output including one or more values of the biometric parameter.

A method including: generating a waveform signal indicative of a biometric parameter of a target subject based at least in part on motion data associated with the target subject; setting a threshold condition based at least in part on one or more characteristics of the waveform signal; and outputting a control signal to a stimulation device based at least in part on the waveform signal satisfying the threshold condition.

Any of the aspects herein, further including: collecting data associated with the target subject, wherein collecting the data includes: receiving, from a depth sensing device, the motion data associated with the target subject.

Any of the aspects herein, wherein collecting the data includes: comparing the one or more characteristics of the waveform signal to a target set of criteria, wherein setting the threshold condition includes maintaining or modifying the threshold condition based at least in part on result of the comparison.

A system including: a stimulation device; a processor; and a memory storing data thereon that, when processed by the processor, cause the processor to: generate a waveform signal indicative of a biometric parameter of a target subject based at least in part on motion data associated with the target subject; set a threshold condition based at least in part on one or more characteristics of the waveform signal; and output a control signal to a stimulation device based at least in part on the waveform signal satisfying the threshold condition.

Any of the aspects herein, further including: a depth sensing device, wherein the instructions are further executable by the processor to: collect data associated with the target subject, wherein collecting the data includes: receive, from the depth sensing device, the motion data associated with the target subject.

Any of the aspects herein, wherein the instructions are further executable by the processor to: compare one or more characteristics of the waveform signal to a set of criteria, wherein setting the threshold condition includes maintaining or modifying the threshold condition based at least in part on a result of the comparison.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The details of one or more aspects of the disclosure are set forth in the accompanying drawings and the description below. Other features, objects, and advantages of the techniques described in this disclosure will be apparent from the description and drawings, and from the claims.

The preceding is a simplified summary of the disclosure to provide an understanding of some aspects of the disclosure. This summary is neither an extensive nor exhaustive overview of the disclosure and its various aspects, implementations, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, implementations, and configurations of the disclosure are possible utilizing, alone or in combination, one or more of the features set forth above or described in detail below.

Numerous additional features and advantages of the present disclosure will become apparent to those skilled in the art upon consideration of the implementation descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, implementations, and configurations of the disclosure, as illustrated by the drawings referenced below.

FIGS. 1A through 1C illustrate examples of a system according to at least one implementation of the present disclosure.

FIGS. 2A and 2B illustrates example visualizations that support aspects of the present disclosure.

FIGS. 3A through 3F illustrate example visualizations that support aspects of the present disclosure.

FIGS. 4A through 4C illustrate example visualizations that support aspects of the present disclosure.

FIGS. 5A through 5C illustrate example visualizations that support aspects of the present disclosure.

FIG. 6 illustrates an example visualization that supports aspects of the present disclosure.

FIGS. 7A and 7B illustrate example visualizations that support aspects of the present disclosure.

FIG. 8 illustrates an example visualization that supports aspects of the present disclosure.

FIG. 9 illustrates an example visualization that supports aspects of the present disclosure.

FIG. 10 illustrates an example of a process flow in accordance with aspects of the present disclosure.

FIGS. 11A through 11C illustrates example visualizations that support aspects of the present disclosure.

### DETAILED DESCRIPTION

It should be understood that various aspects disclosed herein may be combined in different combinations than the combinations specifically presented in the description and accompanying drawings. It should also be understood that, depending on the example or implementation, certain acts or events of any of the processes or methods described herein may be performed in a different sequence, and/or may be added, merged, or left out altogether (e.g., all described acts or events may not be necessary to carry out the disclosed techniques according to different implementations of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10× Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any implementations of the disclosure are explained in detail, it is to be understood that the disclosure is not limited in its application to the details of construction and the arrangement of components set forth in the following description or illustrated in the drawings. The disclosure is capable of other implementations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Aspects of the present disclosure support non-contact patient monitoring. The present disclosure supports non-contact patient monitoring associated with treating and/or diagnosing sleep related disorders (e.g., night tremors, sleep apnea, etc.) and other medical conditions. For example, aspects of the present disclosure support treating and/or diagnosing medical conditions such as mental health conditions (also referred to herein as psychological conditions) and physiological conditions.

A monitoring system described herein provides non-contact, real-time monitoring of respiration or motion of a subject (e.g., a patient) via a depth sensing camera system and software. The monitoring system may be configured to collect motion data and/or depth data associated with the subject. In some aspects, the monitoring system may refrain from collecting video data (e.g., a video feed) of the subject and/or an environment associated with the subject, which may support improved privacy. For example, collecting motion data and/or depth data, without collecting video data, may minimize any concerns associated with having a camera in the subject's environment (e.g., home, hospital room, vehicle, workplace, etc.).

Aspects of the present disclosure support a camera system (e.g., a depth sensing camera system, depth sensing cameras, etc.) for patients experiencing a medical condition for which symptoms are detectable via a change in breathing or bodily motion. For example, aspects of the camera system may support non-contact monitoring of sleep related disorders (e.g., night tremors), physiological conditions (e.g., asthma, respiratory disorders, etc.), and mental health conditions (e.g., anxiety, dementia, depression, etc.).

In some aspects, the camera system may be placed in the environment of a patient to collect data (e.g., motion data, depth data) about the subject over time. Non-limiting examples of the environment include a home of the subject, a bedroom of the subject, a hospital room in which the subject is staying, an office of the subject, and the interior of a vehicle of the subject, and are not limited thereto. The camera system may provide the collected data to the monitoring system. In some aspects, the camera system may provide collected data (e.g., collected quantitative data) to the monitoring system periodically (e.g., based on a schedule and/or trigger condition). Additionally, or alternatively, the camera system may provide collected data (e.g., partially collected data) to the monitoring system in real-time.

In an example, the monitoring system may detect (e.g., based on collected data provided by the camera system) that the subject has exhibited an increased level of motion (e.g., increased respiration rate, rapid breathing, large scale or rapid bodily movements, amount of bodily movement, etc.) that exceeds a pre-established threshold. The monitoring system may provide a response tailored for a therapy or need specific to the subject. For example, if the monitoring system determines that the increased level of motion is indicative of a potential night terror, the monitoring system may respond by generating and/or outputting an alert to wake the subject and end the episode. For example, the monitoring system may generate and/or output an audible alert (e.g., noise), a haptic alert (e.g., vibration), a visual alert (e.g., blinking light), etc. In some examples, the monitoring system may provide a stimulation signal to the subject via a stimulation device (e.g., a neurostimulation device).

The monitoring system supports setting or modifying a stimulation threshold (also referred to herein as a threshold condition) associated with providing therapy to the subject. For example, the monitoring system may set the stimulation threshold in association with achieving a target waveform (e.g., achieving a target morphology of the waveform). The target waveform may be associated with a reduction in severity of or cessation of a medical condition (e.g., occurrence of night terrors, apnea events, etc.). Example aspects of setting or modifying the stimulation threshold are later described herein.

Additionally, or alternatively, the monitoring system may support passive monitoring. For example, the monitoring system may support implementations in which the monitoring system refrains from interacting with (e.g., alerting) a subject. For example, the monitoring system may support recording/monitoring data and providing the same to a healthcare provider (e.g., a physician) or other entity, without interacting with the subject.

Aspects of the non-contact monitoring system described herein may be applied to numerous applications associated with providing medical therapy to a subject. In some cases, aspects of the non-contact monitoring system may be integrated into one or more full systems or therapy solutions/products.

As described herein, aspects of the present disclosure support sensing physiological and/or contextual patient information at a distance. Non-contact monitoring using depth sensing described herein may support touchless monitoring. Aspects of non-contact monitoring described herein may be integrated with a platform technology supported by the monitoring system. For example, the monitoring system may support a layering of features associated with monitoring, detecting, and/or treating medical conditions.

In an example, features associated with monitoring, detecting, and/or treating medical conditions, as supported by the monitoring system, may include respiratory visualization and respiratory waveforms. In some aspects, the features may include monitored and/or generated features such as respiratory rate, presence in bed (AI), and posture (AI). In some other aspects, the features may include central apnea, respiratory patterns, patient activity, and patient identification confirmation. In some aspects, the features may include alarm management, fall detection, vent sedation-agitation, vent asynchrony, tidal volume trending, effort to breathe/obstructive apnea, and respiratory failure prediction.

Physiological information provided by the monitoring system may include, for example, respiratory rate, central apnea, respiratory patterns, vent sedation-agitation, vent asynchrony, tidal volume trending, effort to breathe/obstructive apnea, and respiratory failure, but is not limited thereto. Contextual information provided by the monitoring system may include, for example, presence in bed, posture, patient activity, bed exiting behavior, patient identification confirmation, and fall detection, but is not limited thereto.

In some aspects, the monitoring system may support waiting until a total amount of collected data (e.g., depth data, motion data) associated with a target subject is greater than or equal to a threshold amount, before synthesizing the collected data and generating/predicting a feature (e.g., respiratory patterns, etc.).

Implementations of the present disclosure provide technical solutions to one or more of the problems of ensuring privacy in association with monitoring a subject. For example, aspects of the camera system may support monitoring of sleep related disorders (e.g., night tremors, sleep apnea, etc.), physiological conditions (e.g., asthma, respiratory disorders, etc.), and mental health conditions (e.g., anxiety, dementia, etc.) through the use of motion data and/or depth data, without collecting video data of the subject and/or an environment associated with the subject. In some other aspects, compared to some other systems that rely on captured video data (e.g., red green blue (RGB) video images) in association with monitoring a subject, use of motion data and/or depth data as described herein may provide increased accuracy. In some additional aspects, the techniques described herein provide technical solutions to reducing occurrence of medical events (e.g., apnea events) by setting or modifying a stimulation threshold as described herein.

Aspects of the present disclosure may support implementations of the monitoring system in a hospital environment, a home environment, and other environments (e.g., inside a vehicle). For example, the monitoring system may support respiratory rate monitoring that is continuous, accurate, and everywhere. In some aspects, the monitoring system is highly accurate, continuous showing trends, and supports automatically adding to early warning scores.

The monitoring system may support breathing visualization and respiratory waveforms (e.g., real-time breathing visualization, remote monitors, etc.). In some aspects, aspects of the monitoring system provide improved accuracy compared to manual techniques associated with respiratory rate monitoring (e.g., spot checks by medical personnel).

In another example, the monitoring system may support respiratory surveillance related to post-surgical respiratory compromise, recovery room, post anesthesia care unit (PACU) respiratory compromise surveillance, opioids, and respiratory patterns and apnea. In some aspects, the monitoring system may support respiratory surveillance related to patient controlled analgesia (PCA), gingival crevicular fluid (GCF), and providing relatively quick detection of respiratory failure and a corresponding alarm.

In another example, the monitoring system may support hospital and home sleep monitoring. For example, the monitoring system may support sleep apnea/hypopnea monitoring (e.g., at a sleep lab, general care facility, home monitoring, etc.)

In another example, the monitoring system may support implementations associated with ventilators. For example, the monitoring system may support sedation/agitation monitoring, asynchrony & post-vent weaning (e.g., at an intensive care unit).

In some other examples, the monitoring system may support newborn intensive care unit (NICU) monitoring. For example, the monitoring system may support respiratory rate, apnea detection, activity, A-B-D monitoring, or the like. in some aspects, the monitoring system may support home monitoring of high acuity babies discharged to the home (e.g., monitoring for deteriorations, apnea alert, activity levels, etc.).

Example aspects of implementations of the monitoring system (e.g., in a hospital environment, a home environment, and other environments) are described herein with respect to the following figures.

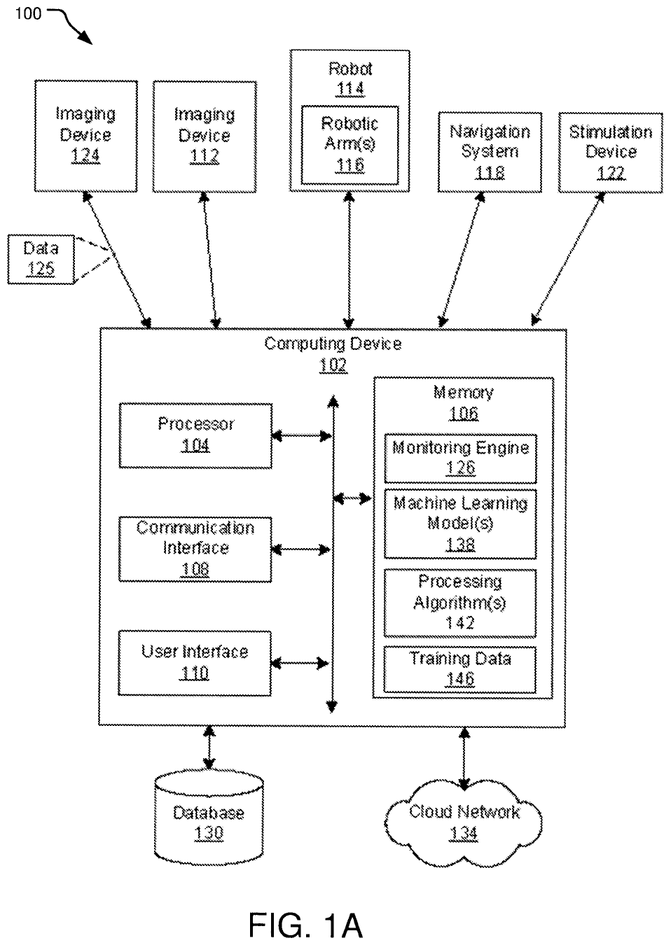

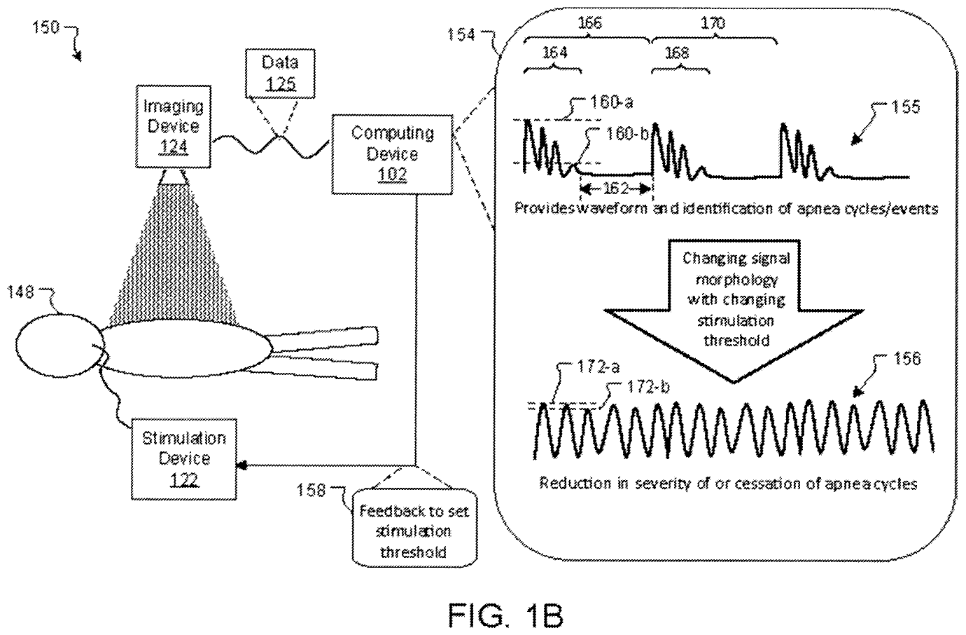

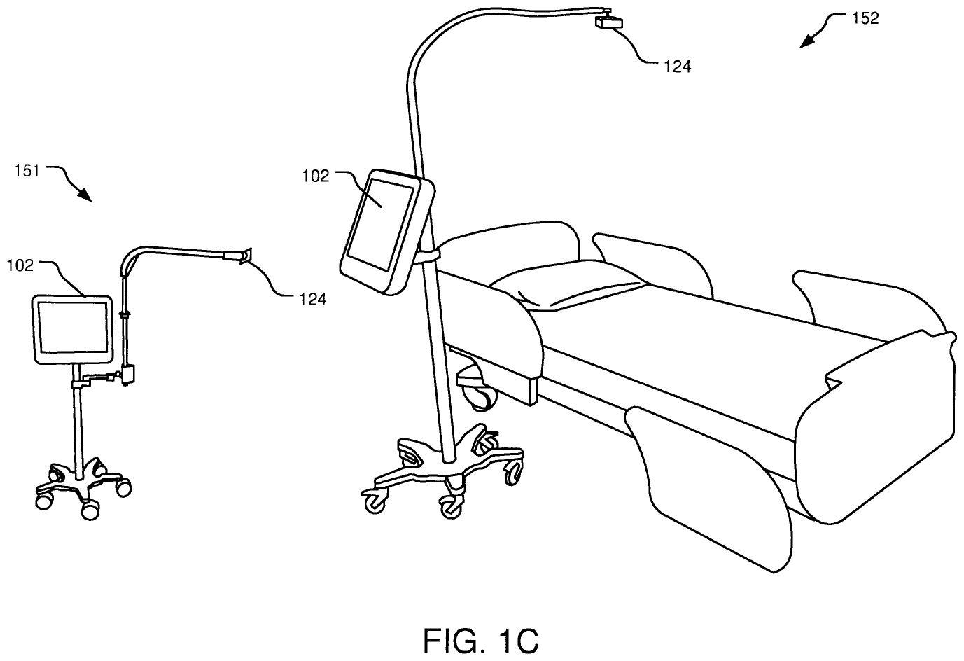

FIG. 1A illustrates an example of a system 100 that supports aspects of the present disclosure. The system 100 may be a monitoring system (e.g., a non-contact monitoring system) supportive of aspects of the present disclosure.

The system 100 includes a computing device 102, imaging device 112, a robot 114, a navigation system 118, a stimulation device 122, imaging device 124, a database 130, and/or a cloud network 134 (or other network). Systems according to other implementations of the present disclosure may include more or fewer components than the system 100. For example, the system 100 may omit and/or include additional instances of the imaging device(s) 112, robot 114, navigation system 118, stimulation device 122, imaging device 124, one or more components of the computing device 102, the database 130, and/or the cloud network 134. In an example, the system 100 may omit any instance of the imaging device(s) 112, robot 114, navigation system 118, stimulation device 122, imaging device 124, one or more components of the computing device 102, the database 130, and the cloud network 134. For example, aspects of the present disclosure support implementing the system 100 without the robot 114 and the navigation system 118. The system 100 may support the implementation of one or more other aspects of one or more of the methods disclosed herein.

The computing device 102 includes a processor 104, a memory 106, a communication interface 108, and a user interface 110. Computing devices according to other implementations of the present disclosure may include more or fewer components than the computing device 102. The computing device 102 may include, for example, electronic circuitry supportive of non-contact, real-time monitoring of respiration or motion of a subject (e.g., a patient) via a depth sensing camera system (e.g., a depth sensing camera device such as imaging device 124, aspects of which are later described herein) and software. The computing device 102 may support generating and outputting a response (e.g., via a user interface 110, via the stimulation device 122, etc.) tailored for providing therapy and/or alerts specific to the subject.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from components (e.g., imaging device 112, robot 114, navigation system 118, stimulation device 122, imaging device 124, database 130, cloud network 134, etc.) of the system 100.

The memory 106 may be or include RAM, DRAM, SDRAM, other solid-state memory, any memory described herein, or any other tangible, non-transitory memory for storing computer-readable data and/or instructions. The memory 106 may store information or data associated with completing, for example, any step of the method 400 described herein, or of any other methods. The memory 106 may store, for example, instructions and/or machine learning models (e.g., machine learning model(s) 138) that support one or more functions of the imaging device 112, robot 114, navigation system 118, stimulation device 122, imaging device 124, and/or the computing device 102. For instance, the memory 106 may store content (e.g., instructions and/or machine learning model(s) 138) that, when executed by the processor 104, enable subject monitoring, data analysis, and notification generation (e.g., by a monitoring engine 126). Such content, if provided as in instruction, may, in some implementations, be organized into one or more applications, modules, packages, layers, or engines.

Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging devices (e.g., imaging device 112, imaging device 124, etc.), the robot 114, navigation system 118, stimulation device 122, the database 130, and/or the cloud network 134.

The computing device 102 may also include a communication interface 108. The communication interface 108 may be used for receiving data or other information from an external source (e.g., imaging device 112, robot 114, navigation system 118, stimulation device 122, imaging device 124, database 130, cloud network 134, and/or any other system or component separate from the system 100), and/or for transmitting instructions, data (e.g., measurements, depth information, motion information, etc.), or other information to an external system or device (e.g., another computing device 102, imaging device 112, robot 114, navigation system 118, stimulation device 122, imaging device 124, database 130, the cloud network 134, and/or any other system or component not part of the system 100). The communication interface 108 may include one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some implementations, the communication interface 108 may support communication between the device 102 and one or more other processors 104 or computing devices 102, whether to reduce the time needed to accomplish a computing-intensive task or for any other reason.

The computing device 102 may also include one or more user interfaces 110. The user interface 110 may be or include a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some implementations, the user interface 110 may support user modification (e.g., by a surgeon, medical personnel, a patient, etc.) of instructions to be executed by the processor 104 according to one or more implementations of the present disclosure, and/or to user modification or adjustment of a setting of other information displayed on the user interface 110 or corresponding thereto.

In some implementations, the computing device 102 may utilize a user interface 110 that is housed separately from one or more remaining components of the computing device 102. In some implementations, the user interface 110 may be located proximate one or more other components of the computing device 102, while in other implementations, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some implementations, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some implementations, the imaging device 112 may comprise more than one imaging device 112. For example, a first imaging device may provide first image data and/or a first image, and a second imaging device may provide second image data and/or a second image. In still other implementations, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some implementations, the robot 114 may be configured to hold and/or manipulate an anatomical element during or in connection with a surgical procedure. The robot 114 may comprise one or more robotic arms 116. In some implementations, the robotic arm 116 may comprise a first robotic arm and a second robotic arm, though the robot 114 may comprise more than two robotic arms. In some implementations, one or more of the robotic arms 116 may be used to hold and/or maneuver the imaging device 112. In implementations where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm).

In some implementations, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some implementations, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some implementations, the navigation system 118 may comprise one or more electromagnetic sensors. In various implementations, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some implementations, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The imaging device 124 may be, for example, a depth camera supportive of object sensing and/or depth sensing. For example, the imaging device 124 may facilitate recognition, registration, localization, mapping, and/or tracking of objects (e.g., patient anatomical features, surgical or other medical instruments, etc.). The imaging device 124 may support recognition and/or tracking of a subject (e.g., patient, healthcare personnel, etc.). For example, the imaging device 124 may identify points in space corresponding to an object (e.g., a patient, an anatomical element of the patient, etc.) based on the distance the point is from the imaging device 124 (e.g., a depth differential between the object and the imaging device 124). The imaging device 124 may support locating objects based on depth, for example, in real-time or near real-time. The imaging device 112 may support facial detection and associated object tracking (e.g., gaze detection, etc.).

The imaging device 124 (e.g., with or without the computing device 102) may process captured data (e.g., depth data) in real-time or near real-time and provide motion data based on data captured at different temporal instances. Accordingly, for example, the imaging device 124 may support motion sensing. For example, the imaging device 124 may track the movement of a subject and/or other objects in the field of view of the imaging device 124. In some aspects, the imaging device 124 may support registry and/or tracking of an object relative to a topography of the subject. The imaging device 124 may provide data 125 to the computing device 102. The data 125 may include depth data and/or motion data described herein.

In some aspects, the imaging device 124 may be integrated with or separate from the imaging device 112. Aspects of manipulating, tracking, and positioning the imaging device 112 (e.g., using the robot 114, robotic arm 116, and/or navigation system 118) described herein may be applied to the imaging device 124.

The stimulation device 122 may be a neurostimulation device (e.g., a neurostimulator) including stimulation circuitry, sensing circuitry, and a stimulation controller (not illustrated). In some examples, the stimulation device 122 may be an implanted neurostimulator (e.g., an implanted pulse generator (IPG))). In some cases, the stimulation device 122 may be a cardiac pacemaker, a cardioverter-defibrillator, a drug delivery device, a biologic therapy device, a monitoring or therapeutic device, etc. In some alternative aspects, the stimulation device 122 may be external to the subject. In some examples, the stimulation device 122 may include aspects (e.g., components, functionalities, etc.) of the computing device 102.

The monitoring engine 126 may generate clinical data associated with a target subject based on the data 125 (e.g., motion data and/or depth data) associated with the target subject. The data 125 may be free of image data (e.g., substantially free of image data). For example, the data 125 may include motion data and/or depth data, without including image data. Additionally, or alternatively, the monitoring engine 126 may generate the clinical data based on the data 125 (e.g., motion data and/or depth data) and image data (e.g., image data captured by the imaging device 112).

In some cases, the monitoring engine 126 may refrain from collecting image data (e.g., image data captured by the imaging device 112). In some other cases, the monitoring engine 126 may utilize the image data (e.g., in combination with the data 125 in association with generating clinical data) but refrain from reporting the image data. For example, the monitoring engine 126 may refrain from providing the image data to another computing device 102, the database 130, the cloud network 134, etc.

The monitoring engine 126 may perform at least one action based on generating the clinical data. For example, the monitoring engine 126 may provide at least a portion of the clinical data to a medical provider. In another example, the monitoring engine 126 may output a notification (e.g., via user interface 100) based on at least the portion of the clinical data satisfying one or more criteria. In some other aspects, based on at least the portion of the clinical data satisfying one or more criteria, the monitoring engine 126 may generate and provide a stimulation signal to the subject (e.g., via stimulation device 122).

The processor 104 may utilize data stored in memory 106 as a neural network. The neural network may include a machine learning architecture. In some aspects, the neural network may be or include one or more classifiers. In some other aspects, the neural network may be or include any machine learning network such as, for example, a deep learning network, a convolutional neural network, a reconstructive neural network, a generative adversarial neural network, or any other neural network capable of accomplishing functions of the computing device 102 described herein. Some elements stored in memory 106 may be described as or referred to as instructions or instruction sets, and some functions of the computing device 102 may be implemented using machine learning techniques.

For example, the processor 104 may support machine learning model(s) 138 which may be trained and/or updated based on data (e.g., training data 146) provided or accessed by any of the computing device 102, the imaging device 112, the robot 114, the navigation system 118, the stimulation device 122, the imaging device 124, the database 130, and/or the cloud network 134. The machine learning model(s) 138 may be built and updated by the monitoring engine 126 based on the training data 146 (also referred to herein as training data and feedback).

For example, the machine learning model(s) 138 may be trained with one or more training sets included in the training data 146. In some aspects, the training data 146 may include multiple training sets. In an example, the training data 146 may include a first training set that includes depth data and/or motion data associated with one or more medical conditions described herein. In an example, the depth data and/or motion data included in the training set may be indicative of changes in breathing or bodily motion indicative of the one or more medical conditions. In some aspects, the depth data and/or motion data included in the training set may be associated with confirmed instances (e.g., by a healthcare provider, a patient, etc.) of the one or more medical conditions.

The training data 146 may include a second training set that includes biometric data (e.g., heart rate, temperature, etc.) associated with one or more medical conditions described herein. In an example, biometric data may be indicative of the one or more medical conditions. In some aspects, the biometric data included in the second training set may be associated with confirmed instances (e.g., by a healthcare provider, a patient, etc.) of the one or more medical conditions.

The training data 146 may include a third training set that includes presence data (e.g., presence in bed), positional data (e.g., posture), and/or behavioral data (e.g., bed exiting behavior) associated with one or more medical conditions described herein. In an example, the presence data, positional data, and/or behavioral data may be indicative of the one or more medical conditions. In some aspects, the presence data, positional data, and/or behavioral data included in the third training set may be associated with confirmed instances (e.g., by a healthcare provider, a patient, etc.) of the one or more medical conditions.

In some examples, based on the data (e.g., depth data, motion data, biometric data, presence data, positional data, behavioral data, etc.), the neural network may generate one or more algorithms (e.g., processing algorithms 142) supportive of non-contact monitoring and detection of patient conditions described herein.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information.

The database 130 may additionally or alternatively store, for example, location or coordinates of devices (e.g., imaging device 112, imaging device 124, stimulation device 122, etc.) of the system 100. The database 130 may be configured to provide any such information to the computing device 102 or to any other device of the system 100 or external to the system 100, whether directly or via the cloud network 134. In some implementations, the database 130 may include treatment information (e.g., for managing a medical condition) associated with a patient. In some implementations, the database 130 may be or comprise part of a hospital image storage system, such as a picture archiving and communication system (PACS), a health information system (HIS), and/or another system for collecting, storing, managing, and/or transmitting electronic medical records including image data.

In some aspects, the computing device 102 may communicate with a server(s) and/or a database (e.g., database 130) directly or indirectly over a communications network (e.g., the cloud network 134). The communications network may include any type of known communication medium or collection of communication media and may use any type of protocols to transport data between endpoints. The communications network may include wired communications technologies, wireless communications technologies, or any combination thereof.

Wired communications technologies may include, for example, Ethernet-based wired local area network (LAN) connections using physical transmission mediums (e.g., coaxial cable, copper cable/wire, fiber-optic cable, etc.). Wireless communications technologies may include, for example, cellular or cellular data connections and protocols (e.g., digital cellular, personal communications service (PCS), cellular digital packet data (CDPD), general packet radio service (GPRS), enhanced data rates for global system for mobile communications (GSM) evolution (EDGE), code division multiple access (CDMA), single-carrier radio transmission technology (1×RTT), evolution-data optimized (EVDO), high speed packet access (HSPA), universal mobile telecommunications service (UMTS), 3G, long term evolution (LTE), 4G, and/or 5G, etc.), Bluetooth®, Bluetooth® low energy, Wi-Fi, radio, satellite, infrared connections, and/or ZigBee® communication protocols.

The Internet is an example of the communications network that constitutes an Internet Protocol (IP) network consisting of multiple computers, computing networks, and other communication devices located in multiple locations, and components in the communications network (e.g., computers, computing networks, communication devices) may be connected through one or more telephone systems and other means. Other examples of the communications network may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VoIP) network, a cellular network, and any other type of packet-switched or circuit-switched network known in the art. In some cases, the communications network 120 may include of any combination of networks or network types. In some aspects, the communications network may include any combination of communication mediums such as coaxial cable, copper cable/wire, fiber-optic cable, or antennas for communicating data (e.g., transmitting/receiving data).

The computing device 102 may be connected to the cloud network 134 via the communication interface 108, using a wired connection, a wireless connection, or both. In some implementations, the computing device 102 may communicate with the database 130 and/or an external device (e.g., a computing device) via the cloud network 134.

The system 100 or similar systems may be used, for example, to carry out one or more aspects of any of the process flow 1000 and process flow 1200 described herein. The system 100 or similar systems may also be used for other purposes.

FIG. 1B illustrates an example 150 of the system 100 that supports aspects of the present disclosure.

In an example, the imaging device 124 may provide data 125 (e.g., motion data and/or depth data) associated with a subject 148 to the computing device 102. Based on the data 125, the computing device 102 may generate and provide a visualization 154 associated with a medical condition. The visualization 154 may include a waveform 155 (also referred to herein as a waveform signal) indicative of a biometric parameter of the target subject 148. In an example, the biometric parameter may be a respiration rate or a tidal volume.

In some cases, the computing device 102 may provide the data 125 (e.g., motion data and/or depth data) to machine learning model 138 (e.g., described with reference to FIG. 1A). The computing device 102 may receive an output from the machine learning model 128 in response to the machine learning model 128 processing the data 125 (or a portion thereof). The output may include one or more values of the biometric parameter (e.g., respiration rate, tidal volume, etc.).

In an example, each rise of the waveform 155 may represent an inhalation of the target subject 148, and each fall of the waveform 155 may represent an exhalation of the target subject 148. Duration 162 may represent a temporal period during which the target subject 148 is not breathing. Based on characteristics of the waveform 155, the computing device 102 may detect various types of respiratory patterns associated with the target subject 148. In some cases, based on the characteristics of the waveform 155, the computing device 102 may identify different medical conditions (e.g., apnea cycles, apnea events, hypopnea cycles, hypopnea events, night tremors, etc.) or different states of the medical conditions.

The computing device 102 may identify a medical condition (e.g., apnea event, hypopnea event, night tremor, etc.) based on pattern 164 of the waveform 155. For example, the computing device 102 may identify the medical condition based on a difference value between an amplitude 160-a (e.g., a maximum amplitude) and an amplitude 160-b (e.g., a minimum amplitude) of the pattern 164. In another example, the computing device 102 may identify the medical condition based on a ratio between amplitude 160-a and amplitude 160-b. In another example, the computing device 102 may identify the medical condition based on pattern 166 of the waveform 155 (e.g., including duration 162 in which the target subject 148 is not breathing). In some cases, the computing device 102 may identify the medical condition based on the quantity of times the pattern 164 or the pattern 166 repeats with respect to a temporal duration.

In some aspects (not illustrated), the visualization 154 may include a visual representation of the motion data and/or the depth data with respect to the target subject 148. For example, the visualization 154 may include a depth image associated with the target subject 148. In some other examples, the visualization 154 may include an RGB image of the target subject 148. In another example, the visualization 154 may include an RGB-D image of the target subject 148 (e.g., a combination of an RGB image and a corresponding depth image). Examples of the visualization 154 are later described herein.

The computing device 102 may calculate a stimulation threshold (also referred to herein as a threshold condition) associated with treating the medical condition. The stimulation threshold may be associated with the biometric parameter (e.g., respiration rate, tidal volume, etc.) represented by the waveform 155. In an example, the stimulation threshold may be, for example, a threshold respiration rate or a threshold tidal volume. The stimulation threshold is not limited thereto, and the stimulation threshold may include alternative and/or additional threshold conditions described herein.

In an example, the computing device 102 may set the stimulation threshold in association with achieving a target waveform 156 (e.g., achieving a morphology of the waveform 156). Target waveform 156 may be associated with a reduction in severity of or cessation of the medical condition. In some examples, the target waveform 156 may indicate a breathing pattern in which inhalations and exhalations represented by the waveform 155 are relatively regular. For example, the target waveform 156 may be free from medical events (e.g., apnea events, hypopnea events, etc.). Example aspects of setting and applying the stimulation threshold in association with achieving target waveform 156 are described herein. In some aspects, a difference between a maximum amplitude 172-a and a minimum amplitude 172-b of the target waveform 156 may be less than a target difference value. In some aspects, a ratio between a maximum amplitude 172-a and a minimum amplitude 172-b of the target waveform 156 may be equal to a target ratio (e.g., within a threshold tolerance).

The computing device 102 may utilize the stimulation threshold in association with providing control signals to the stimulation device 122 (e.g., as feedback 158). For example, if the computing device 102 identifies that a characteristic of the waveform 155 satisfies the stimulation threshold (e.g., based on the morphology of the waveform 155), the computing device 102 may output a control signal to the stimulation device 122. In response to the control signal, the stimulation device 122 may deliver therapy (e.g., electrical stimulation, drug delivery, etc.) to the target subject 148. After the stimulation device 122 has delivered therapy to the target subject 148, the computing device 102 may collect additional data 125 (e.g., motion data, depth data, etc.) from the imaging device 124. Based on the data 125, the computing device 102 may generate additional data for the waveform 155 (e.g., update the waveform 155). The computing device 102 may identify whether the waveform 155 (or updated portion thereof) matches the waveform 156 (e.g., within a threshold tolerance).

Example aspects of iteratively modifying the stimulation threshold in association with achieving target waveform 156 are described herein. For example, aspects of the present disclosure support operations of iteratively modifying the stimulation threshold, outputting control signals to the stimulation device 122 based on the stimulation threshold (e.g., if a characteristic of the waveform 155 satisfies the stimulation threshold), and delivering therapy to the target subject 148 in response to the control signals, until the waveform 155 satisfies a set of criteria (e.g., the waveform 155 matches the target waveform 156 within a threshold tolerance).

In an example implementation of modifying the stimulation threshold, the computing device 102 may compare one or more characteristics of the waveform 155 to a set of criteria. If the criteria is satisfied, the computing device 102 may set (e.g., maintain or modify) the stimulation threshold. The characteristics of the waveform 155 may include a morphology of the waveform 155. In an example, the characteristics (e.g., morphology) may include parameters such as the peak to peak amplitude, the interval between peaks, a number of zero crossings, the maximum slope (positive or negative), a number of peaks or valleys, an area under the curve, or other characteristics of the waveform 155.

In an example implementation, the computing device 102 may calculate a ratio associated with an amplitude 160-a of the waveform 155 and an amplitude 160-b of the waveform 155. The computing device 102 may compare the ratio to a target ratio. If the ratio is equal to the target ratio within a tolerance value, the computing device 102 may maintain the stimulation threshold. Additionally, or alternatively, if the ratio is different from the target ratio by more than the tolerance value, the computing device 102 may modify the stimulation threshold.

In another example implementation, the computing device 102 may calculate a difference value between the amplitude 160-a of the waveform 155 and the amplitude 160-b of the waveform 155. The computing device 102 may compare the difference value to a target difference value. If the difference value is less than the target difference value, the computing device 102 may maintain the stimulation threshold. Additionally, or alternatively, if the difference value is greater than the target difference value, the computing device 102 may modify the stimulation threshold.

In another example implementation, the computing device 102 may calculate the duration 162 between the end of the pattern 164 and the start of another pattern 168. The computing device 102 may compare the duration 162 to a target duration. If the duration 162 is less than the target duration, the computing device 102 may maintain the stimulation threshold. Additionally, or alternatively, if the duration 162 is greater than the target duration, the computing device 102 may modify the stimulation threshold.

In some cases, aspects of the present disclosure support using the characteristics (e.g., ratio between amplitude 160-a and amplitude 160-b, difference value between amplitude 160-a and amplitude 160-b, duration 162, etc.) of the waveform 155 as the stimulation threshold. For example, the stimulation device 122 may deliver therapy to the target subject 148 until the ratio changes (e.g., until a 1:1 ratio is achieved). In another example, the stimulation device 122 may deliver therapy to the target subject 148 until the difference value changes (e.g., until the difference value decreases to a target difference value). In another example, the stimulation device 122 may deliver therapy to the target subject 148 until the duration 162 (e.g., until the duration 162 decreases to the target duration).

In some aspects, based on the waveform 155, the computing device 102 may identify a quantity of apnea events in association with a temporal duration. For example, the computing device 102 may calculate an apnea hypopnea index (AHI) based on the waveform 155. In another example, based on the waveform 155, the computing device 102 may identify a quantity of hypopnea events in association with a temporal duration. For example, the computing device 102 may calculate a respiratory disturbance index (RDI) based on the waveform 155.

The computing device 102 may compare a parameter value associated with apnea events (e.g., quantity of apnea events in association with a temporal duration, AHI, etc.) to a corresponding threshold value. Additionally, or alternatively, the computing device 102 may compare a parameter value associated with hypopnea events (e.g., quantity of hypopnea events in association with a temporal duration, RDI, etc.) to a corresponding threshold value.

In an example implementation, if the parameter value associated with apnea events (e.g., quantity of apnea events, AHI, etc.) is less than the corresponding threshold value, the computing device 102 may maintain the stimulation threshold. Additionally, or alternatively, if the parameter value associated with hypopnea events (e.g., quantity of hypopnea events, RDI, etc.) is less than the corresponding threshold value, the computing device 102 may maintain the stimulation threshold. In another example, if the parameter value associated with apnea events (e.g., quantity of apnea events, AHI, etc.) is equal to or greater than the corresponding threshold value and/or the parameter value associated with hypopnea events (e.g., quantity of hypopnea events, RDI, etc.) is equal to or greater than the corresponding threshold value, the computing device 102 may modify the stimulation threshold.

Aspects of the present disclosure support using parameter values associated with apnea events and/or hypopnea events as the stimulation threshold. For example, the stimulation device 122 may deliver therapy to the target subject 148 until the parameter value associated with apnea events (e.g., quantity of apnea events, AHI, etc.), as determined from the waveform 155, decreases. In another example, the stimulation device 122 may deliver therapy to the target subject 148 until the parameter value associated with hypopnea events (e.g., quantity of hypopnea events, RDI, etc.), as determined from the waveform 155, decreases. In some aspects, the stimulation device 122 may deliver therapy to the target subject 148 and until the quantity of apnea events and/or hypopnea events with respect to a temporal duration reaches zero.

In some alternative or additional implementations, the computing device 102 may receive biometric measurements from sensing devices (e.g., imaging device 112) other than the imaging device 124. Based on the biometric measurements and/or the data 125, the computing device 102 may generate and provide the visualization 154 (and waveform 155) described herein.

FIG. 1C illustrates an example 151 and an example 152 of the system 100 that support aspects of the present disclosure.

Example 151 illustrates an example implementation of the computing device 102 and the imaging device 124. Example 152 illustrates an example implementation of the computing device 102, the imaging device 124, and a hospital environment (e.g., a hospital room). In some aspects, the imaging device 124 may support any combination of form factors (e.g., attached to bed, a headboard, a wall, a freestanding apparatus, etc.).

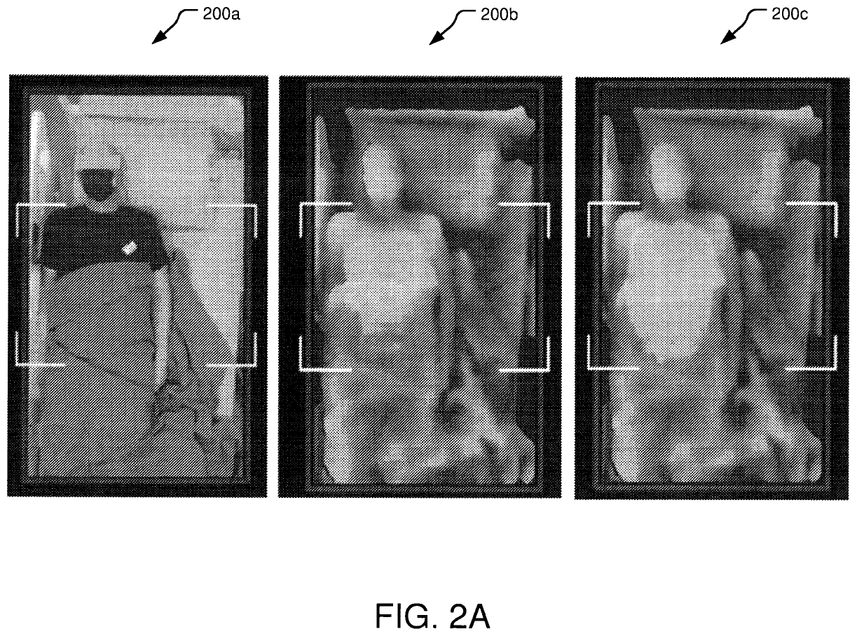

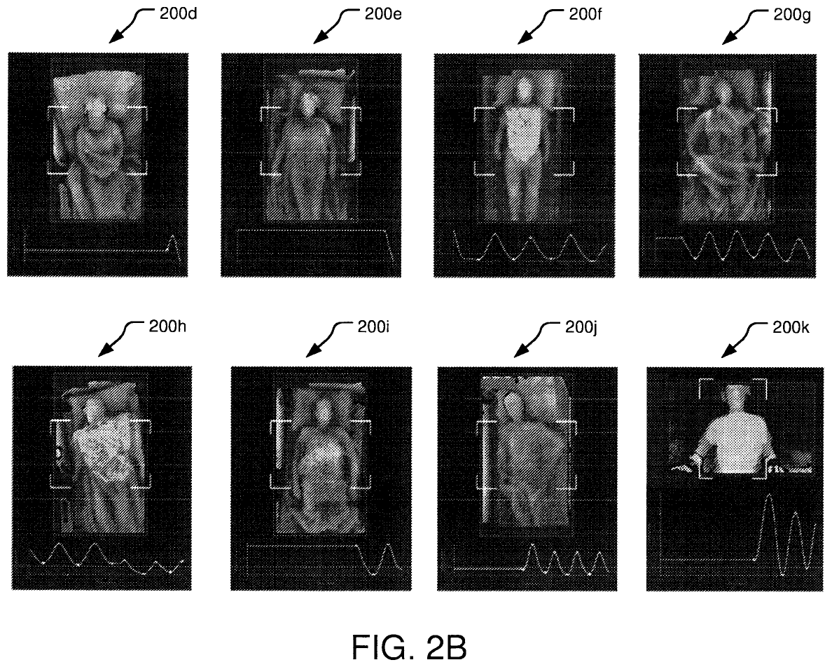

FIGS. 2A and 2B illustrates example visualizations 200-a through 200-k that support aspects of the present disclosure. Visualization 200-a through visualization 200-k may include aspects of a visualization 154 described herein.

Visualization 200-a includes an RGB image including image data (e.g., video image data) of the target subject 148. For example, the computing device 102 may generate the RGB image of visualization 200-a based on image data provided by the imaging device 112.

Visualization 200-b through visualization 200-k include depth images associated with the target subject 148. For example, the computing device 102 may generate the depth images of visualization 200-b through visualization 200-j based on the data 125 (e.g., motion data, depth data) provided by the imaging device 124.

The computing device 102 may support switching between displaying different image types (e.g., an RGB image, a depth image, an RGB-D image, etc.), for example, via the user interface 110.

In some aspects, the computing device 102 may generate and display a respiratory waveform (e.g., as illustrated at visualization 200-d through visualization 200-k) based on the data 125 (e.g., motion data, depth data) provided by the imaging device 124. In an example, the respiratory waveform may include information such as displacement vs. time (seconds). The computing device 102 may generate the respiratory waveform using the processing algorithm(s) 142 described herein. Accordingly, for example, aspects of the present disclosure support providing breathing visualizations associated with a target subject 148.

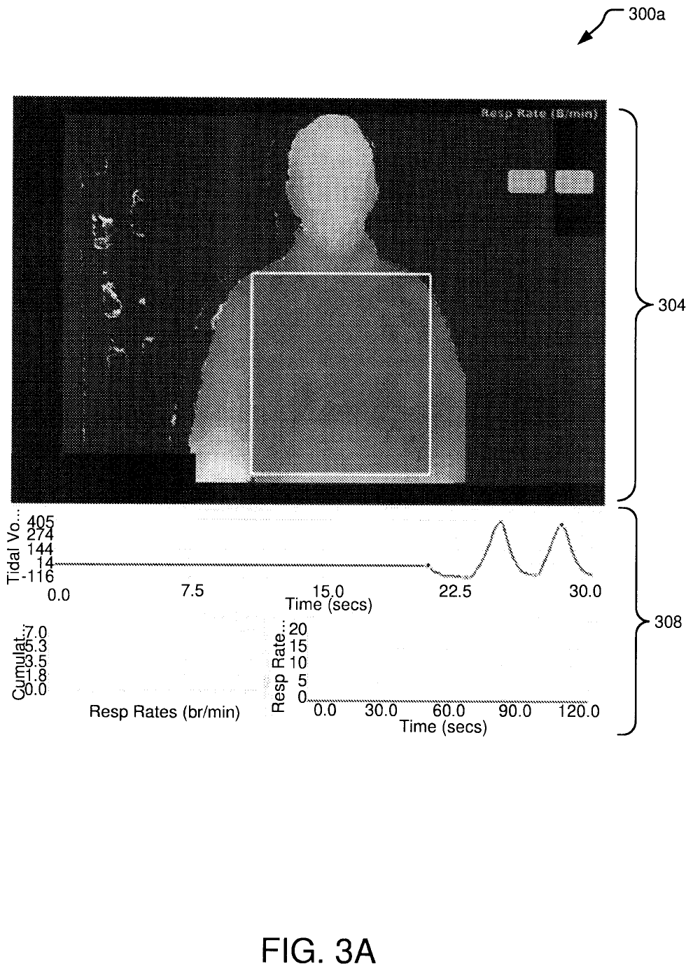

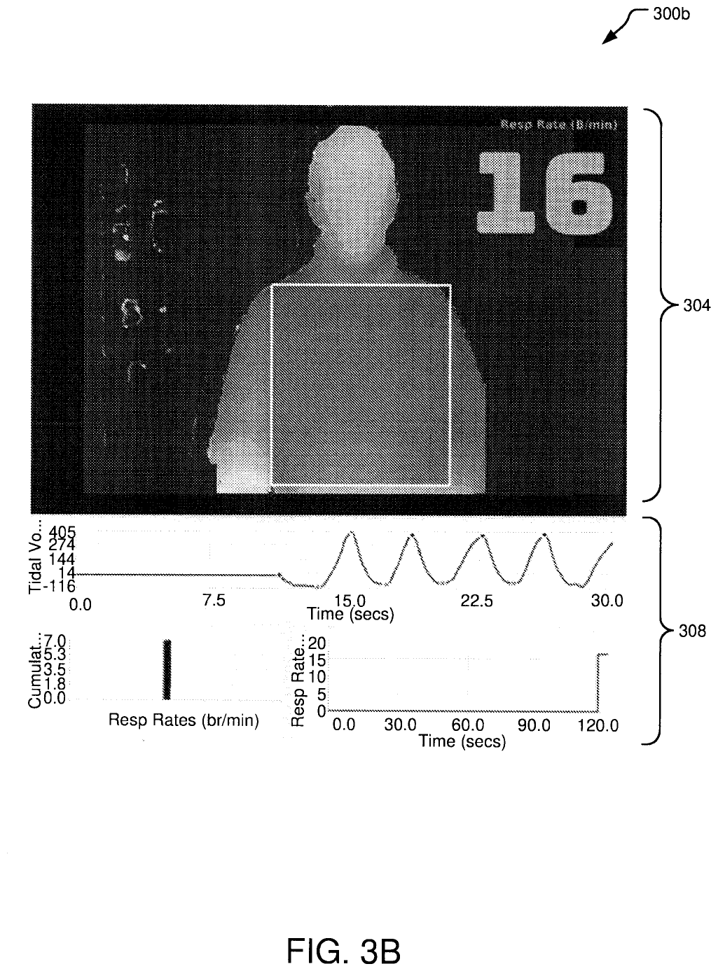

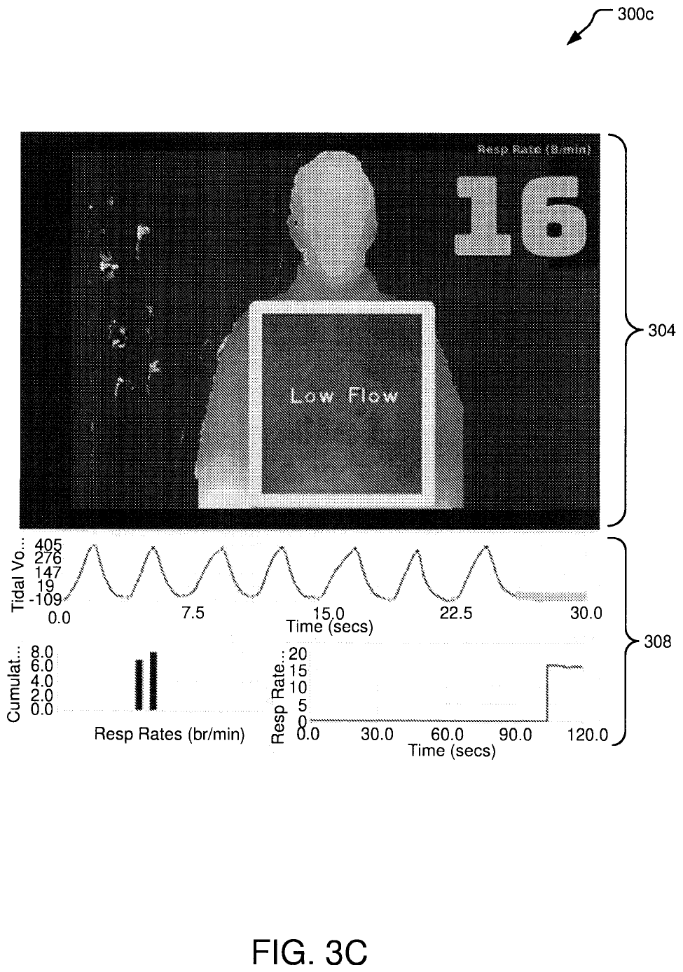

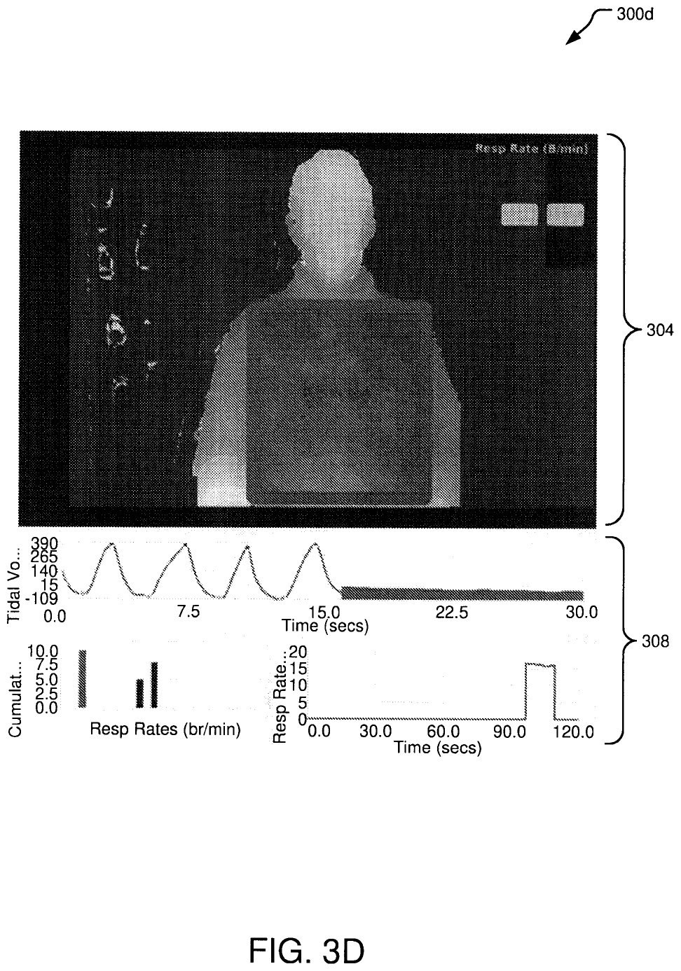

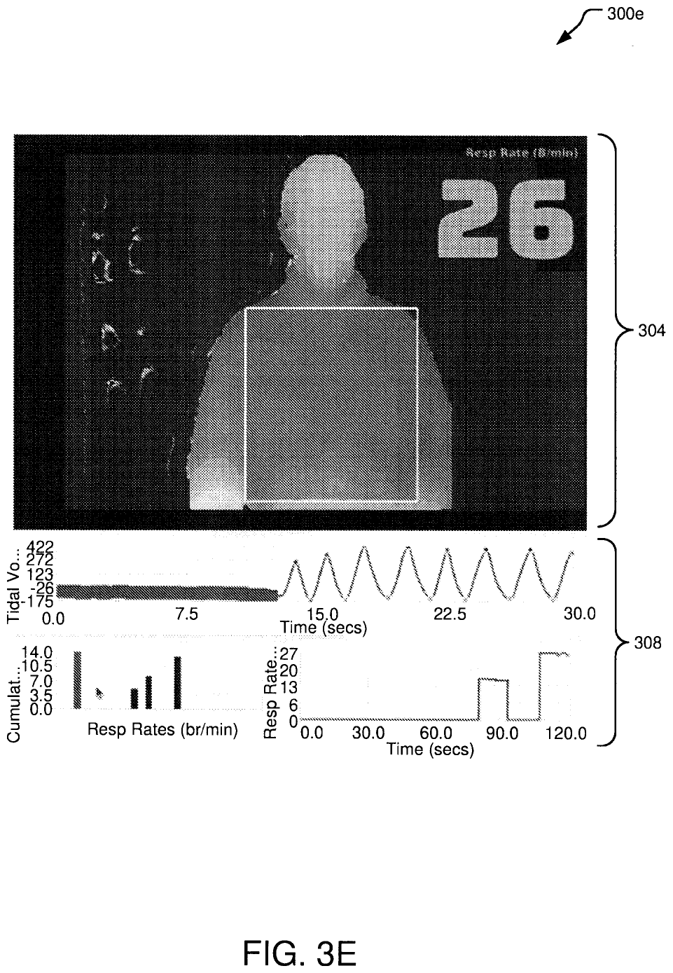

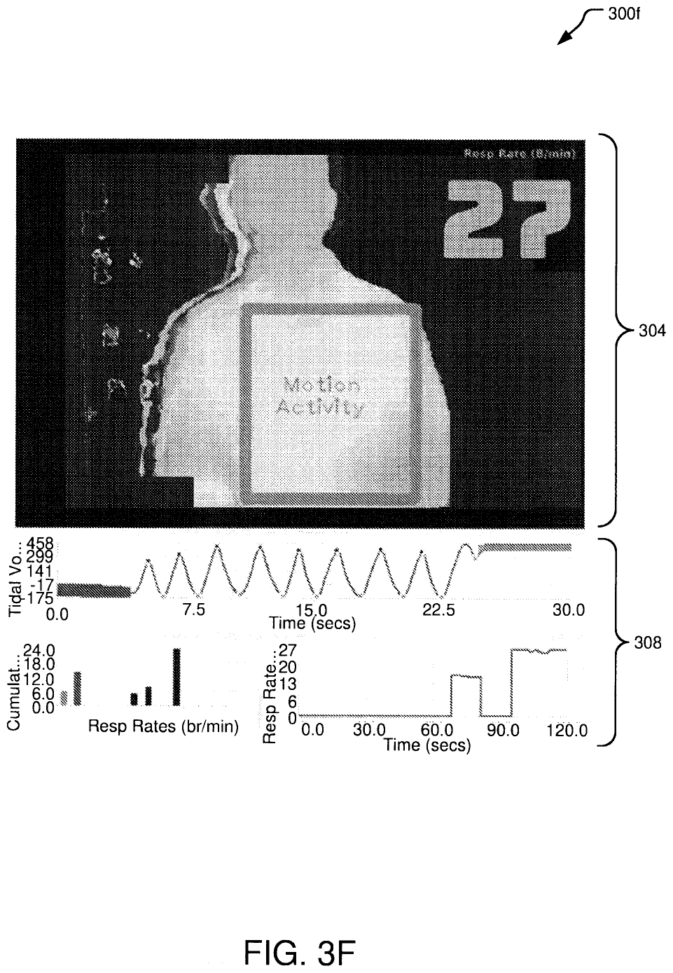

FIGS. 3A through 3F illustrate example visualizations 300-a through 300-e that support aspects of the present disclosure. Visualization 300-a through visualization 300-e may include aspects of a visualization 154 described herein.

Each visualization 300 may include first information 304 and second information 308. First information 304 may include an image (e.g., an RGB image, a depth image, an RGB-D image, etc.) of the target subject 148, an alphanumeric indication representative of biometric information (e.g., respiratory activity, respiratory rate, etc.) associated with the target subject 148, and status information (e.g., a low flow event, an apnea event, motion activity, etc.) associated with the target subject 148. Second information 308 may include a combination of waveforms, graphs, charts, etc. representative of the biometric information (e.g., respiratory activity, respiratory rate, etc.) associated with the target subject 148.

In an example, referring to visualization 300-a, the amount of data 125 (e.g., motion data, depth data, etc.) provided by the imaging device 124 to the computing device 102 may be less than a threshold amount. Accordingly, for example, the computing device 102 may wait until additional amounts of the data 125 are received before calculating a respiratory rate of the target subject 148 (e.g., until a total amount of the data 125 is greater than or equal to a threshold amount).

In an example, referring to visualization 300-b, the first information 304 and second information 308 indicate a respiratory rate of 16 breaths per minute associated with the target subject 148. In another example, referring to visualization 300-c, the first information 304 and second information 308 indicate a respiratory rate of 16 breaths per minute and indicate a low flow (e.g., low airflow) event associated with the target subject 148. In another example, referring to visualization 300-d, the first information 304 and second information 308 indicate an apnea event associated with the target subject 148. In another example, referring to visualization 300-f, the first information 304 and second information 308 indicate a respiratory rate of 27 breaths per minute and indicate a motion event (e.g., motion activity) associated with the target subject 148.

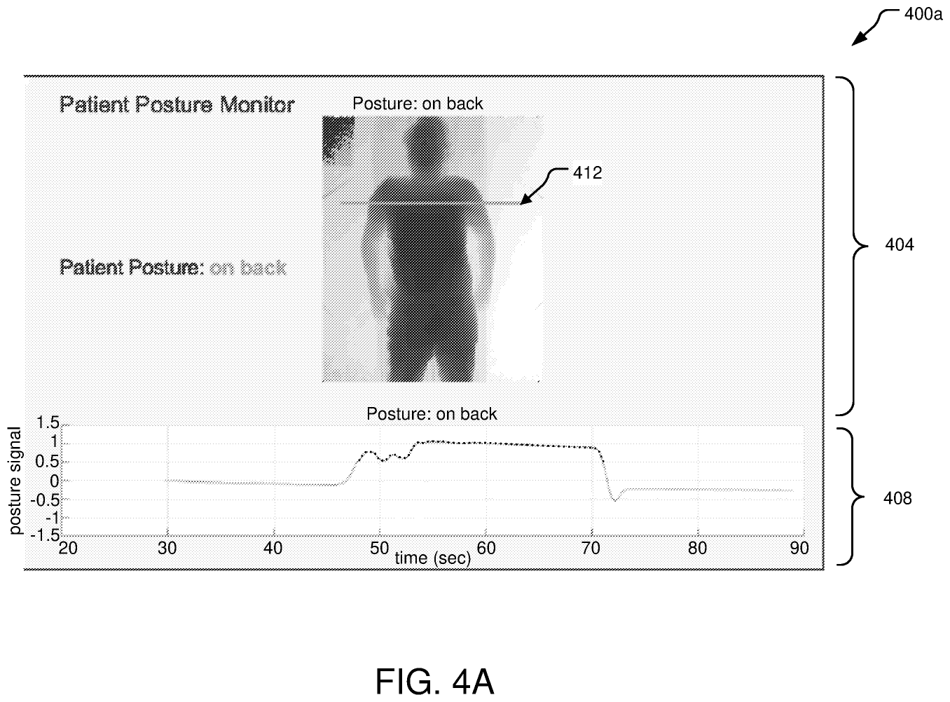

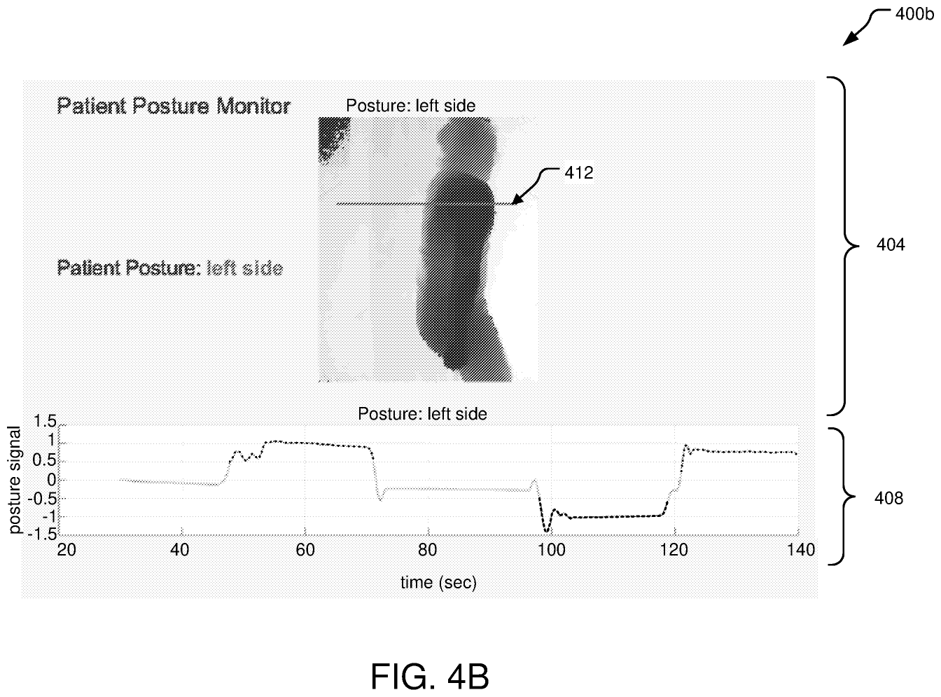

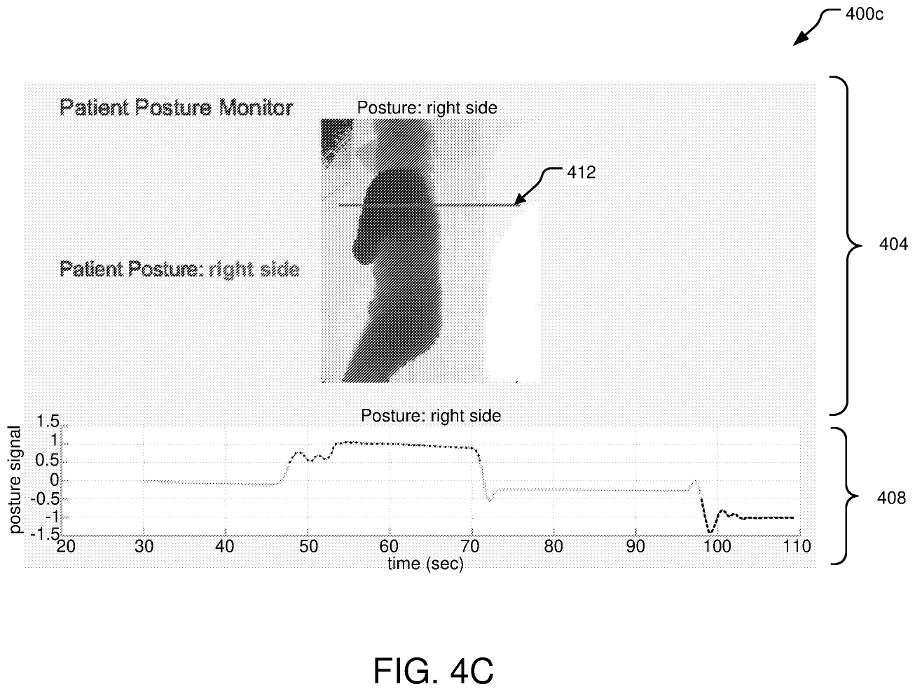

FIGS. 4A through 4C illustrate example visualizations 400-a through 400-c that support aspects of the present disclosure. Visualization 400-a through visualization 400-c may include aspects of a visualization 154 described herein.

Each visualization 400 may include first information 404 and second information 408. First information 404 may include an image (e.g., an RGB image, a depth image, an RGB-D image, etc.) of the target subject 148 and posture information associated with the target subject 148. Second information 408 may include a combination of waveforms, graphs, charts, etc. representative of the posture associated with the target subject 148. For example, the second information 408 may include a graph of a posture signal (e.g., ranging from values of +1.5 to −1.5). The computing device 102 may calculate the posture signal based on data 125 (e.g., motion data, depth data) corresponding to a set of target coordinates (e.g., a target line 412) associated with the target subject 148.

In an example, referring to visualization 400-a, the first information 404 and second information 408 (e.g., a posture signal of about 0) may indicate a patient posture ‘on back’.

In another example, referring to visualization 400-b, the first information 404 and second information 408 (e.g., a posture signal equal to or greater than about +0.5, for example, a posture signal equal to about +1) may indicate a patient posture ‘left side’.

In another example, referring to visualization 400-c, the first information 404 and second information 408 (e.g., a posture signal equal to or less than about −0.5, for example, a posture signal equal to about −1) may indicate a patient posture ‘right side’.

In some aspects, the computing device 102 may wait a total amount of the data 125 is greater than or equal to a threshold amount before synthesizing the data 125 (e.g., predicting or detecting posture).

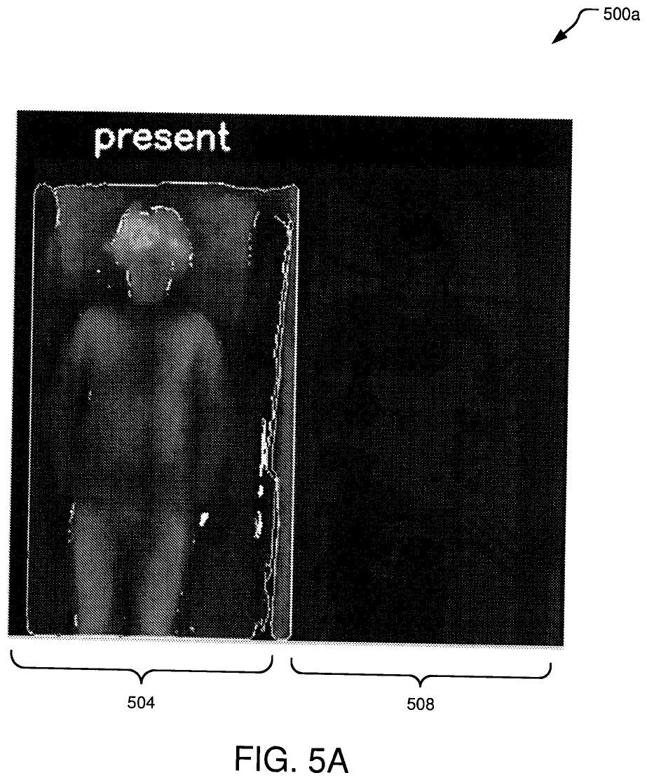

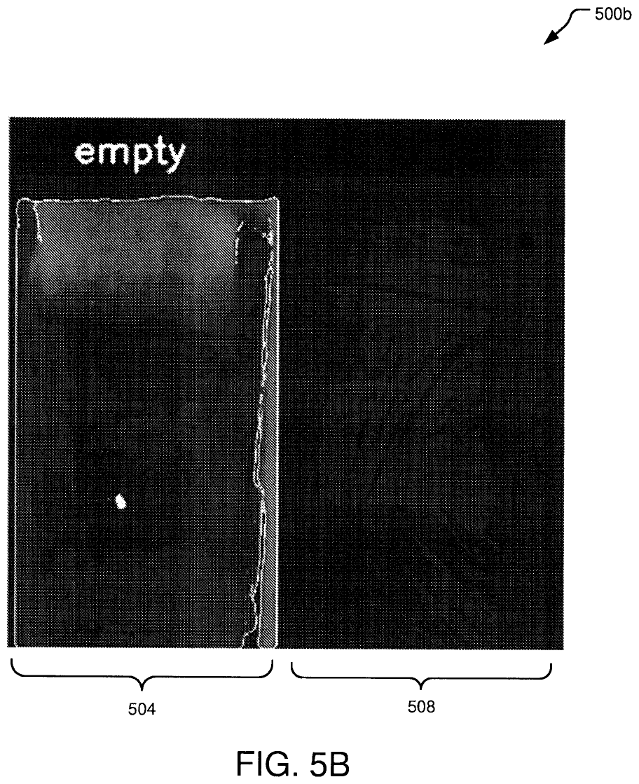

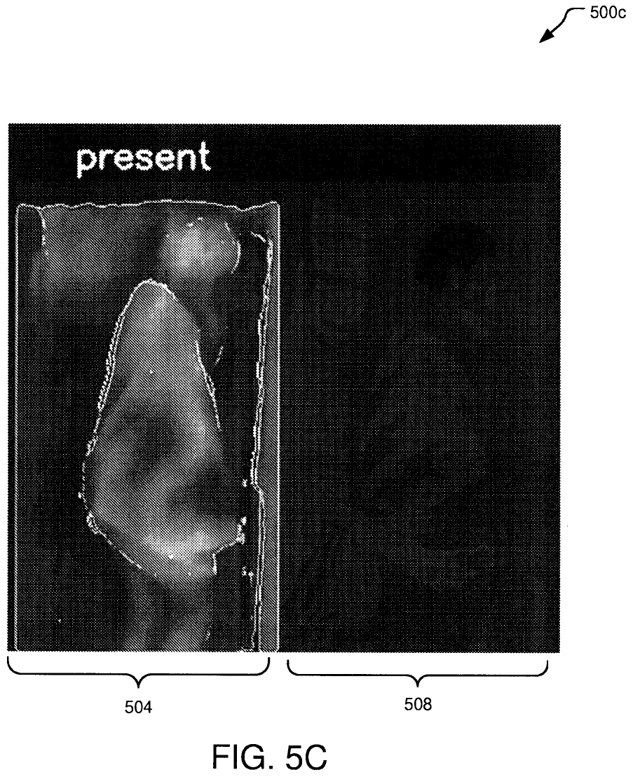

FIGS. 5A through 5C illustrate example visualizations 500-a through 500-c that support aspects of the present disclosure. Visualization 500-a through visualization 500-c may include aspects of a visualization 154 described herein.

Each visualization 500 may include a first image 504 (e.g., a depth image) and a second image 508 (e.g., an RGB image) of the target subject 148. Additionally, or alternatively, each visualization 500 may include a single (e.g., an RGB-D image) of the target subject 148. In some aspects, each visualization 500 may include presence information associated with the target subject 148. The computing device 102 may calculate the presence information based on the data 125 (e.g., motion data, depth data) associated with the target subject 148.

In an example, referring to visualization 500-a, the first image 504 and/or the second image 508 may indicate the target subject 148 is ‘present’. In another example, referring to visualization 500-b, the first image 504 and/or the second image 508 may indicate the target subject 148 is ‘not present’ (e.g., the bed is empty). In another example, referring to visualization 500-c, the first image 504 and/or the second image 508 may indicate the target subject 148 is ‘present’.

In some aspects (not illustrated), each visualization 500 may include respiratory rate and/or posture information as described herein.

In some aspects, the computing device 102 may wait a total amount of the data 125 is greater than or equal to a threshold amount before synthesizing the data 125 (e.g., predicting or detecting presence of the target subject 148).

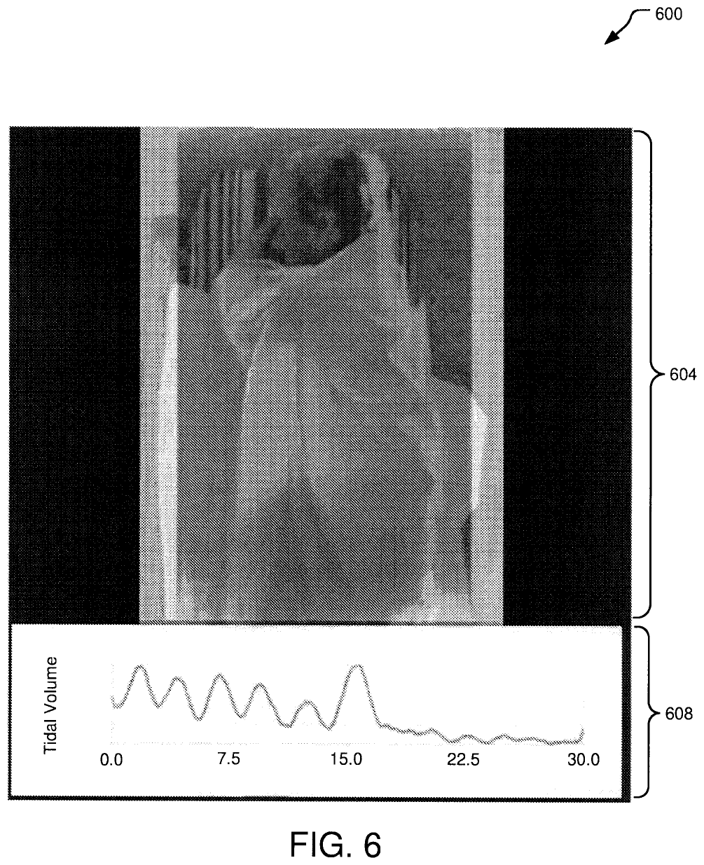

FIG. 6 illustrates an example visualization 600 that supports aspects of the present disclosure. Visualization 600 may include aspects of a visualization 154 described herein. FIG. 6 illustrates aspects associated with monitoring breathing patterns of the target subject 148. In some aspects, the system 100 may support monitoring for abnormal breathing patterns (e.g., Cheyne-Stokes respiration, intermittent breathing, opioids induced respiratory depression, etc.).

Visualization 600 may include first information 604 and second information 608. First information 604 may include an image (e.g., an RGB image, a depth image, an RGB-D image, etc.) of the target subject 148 and volume information associated with the target subject 148. Second information 608 may include a combination of waveforms, graphs, charts, etc. representative of the volume information (e.g., sleep cycles) associated with the target subject 148. For example, the second information 608 may include a graph of a total volume associated with the target subject 148. The computing device 102 may calculate the total volume based on the data 125 (e.g., motion data, depth data) associated with the target subject 148.

Aspects of the present disclosure support providing highly accurate data under various conditions. For example, referring to FIG. 6, the computing device 102 may generate the visualization 600 under conditions in which the target subject 148 is on their side, under an object (e.g., bed covers), and/or in low lighting.

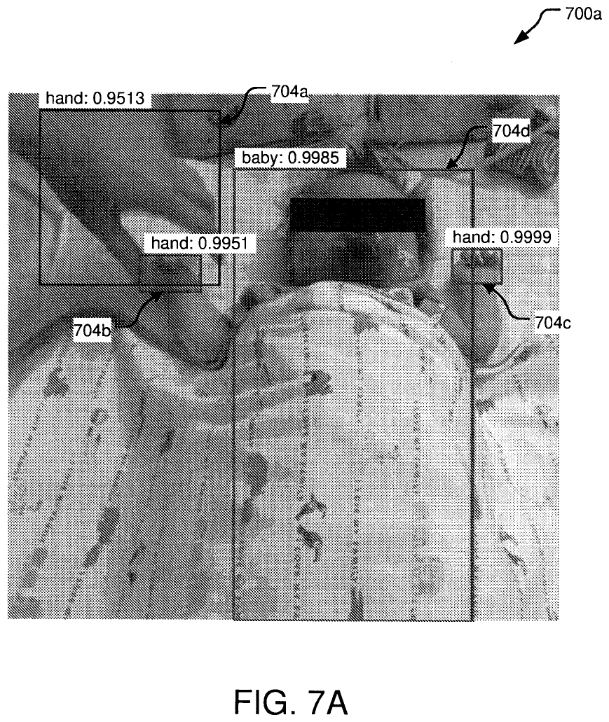

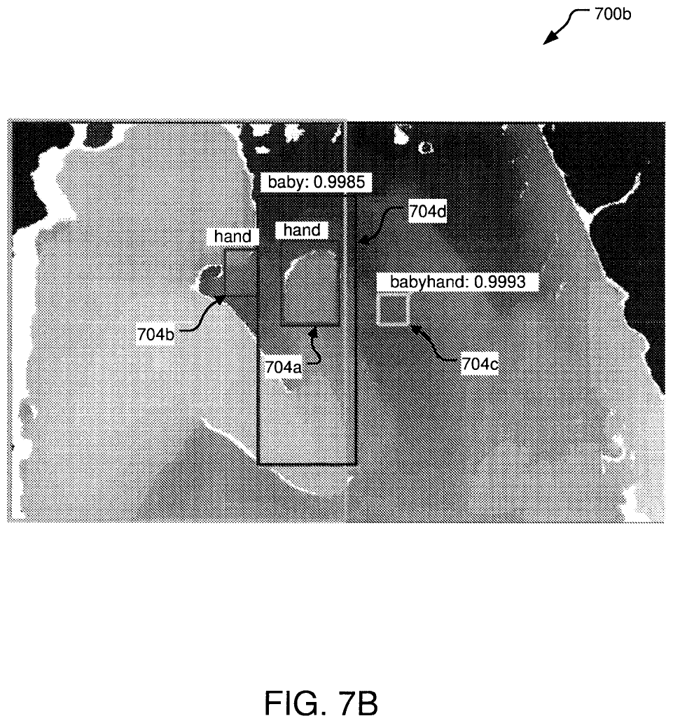

FIGS. 7A and 7B illustrate example visualizations 700-a and 700-b that support aspects of the present disclosure. Visualization 700-a and visualization 700-b illustrate aspects of object detection and/or tracking supported by the system 100, for example, with respect to caregiver interaction. In the examples of FIGS. 7A and 7B, visualization 700 illustrates an RGB image of a target subject 148 (e.g., an infant), and visualization 700-b illustrates a depth image of the target subject 148.

The computing device 102 may generate bounding boxes 704 (e.g., bounding box 704-a through bounding box 704-d) associated with the target subject 148 and a caregiver interacting with the target subject 148. The computing device 102 may generate the bounding boxes 704 based on the data 125 (e.g., motion data, depth data) provided by the imaging device 124. The computing device 102 (e.g., machine learning model(s) 138) may calculate probability scores and/or confidence scores (e.g., having values ranging from 0.00 to 1.00) associated with each of the bounding boxes 704.

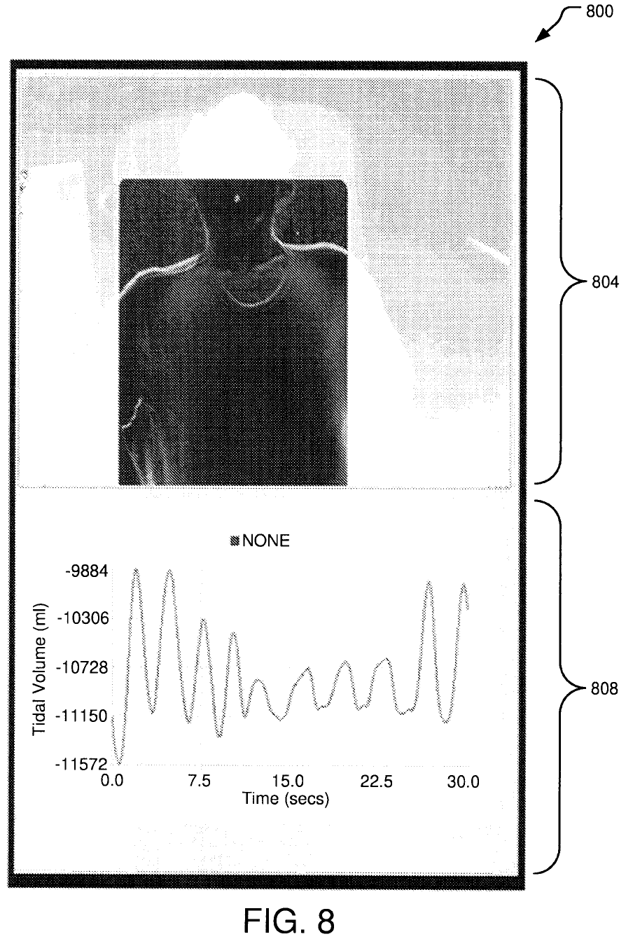

In an example, the computing device 102 may identify that the bounding box 704-a corresponds to a hand of the caretaker (e.g., healthcare provider, medical personnel, a parent, etc.), the bounding box 704-b corresponds to a hand of the target subject 148, the bounding box 704-c corresponds to another hand of the target subject 148, and the bounding box 704-d corresponds to the body and head of the target subject 148 FIG. 8 illustrates an example visualization 800 that supports aspects of the present disclosure. Visualization 800 may include aspects of a visualization 154 described herein. FIG. 8 illustrates aspects associated with monitoring activity associated with an effort to breathe by the target subject 148.

Visualization 800 may include first information 804 and second information 808. First information 804 may include an image (e.g., an RGB image, a depth image, an RGB-D image, etc.) of the target subject 148. Second information 808 may include a combination of waveforms, graphs, charts, etc. representative of the tidal volume (e.g., amount of air that moves in or out of the lungs with each respiratory cycle) associated with the target subject 148. For example, the second information 408 may include a graph of the tidal volume with respect to time for the target subject 148. The computing device 102 may calculate the tidal volume based on the data 125 (e.g., motion data, depth data) associated with the target subject 148.

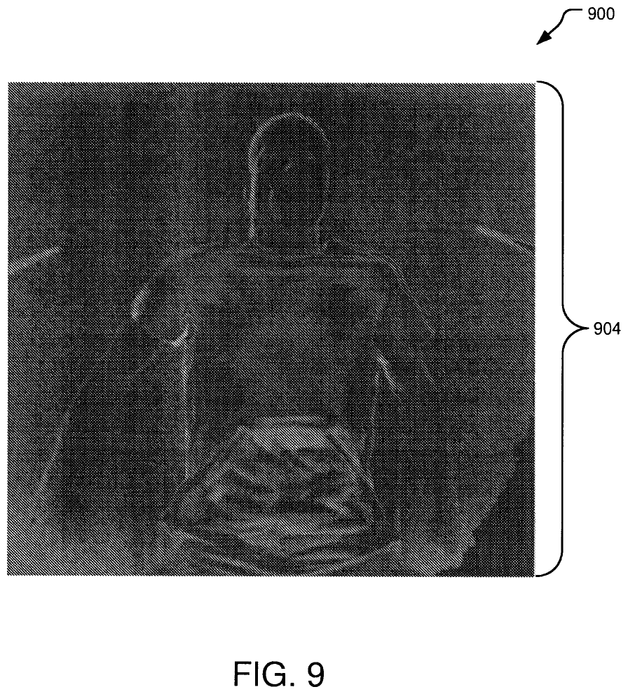

FIG. 9 illustrates an example visualization 900 that supports aspects of the present disclosure. Visualization 900 may include aspects of a visualization 154 described herein. FIG. 9 illustrates aspects associated with monitoring activity associated with obstructive apnea experienced by the target subject 148.

Visualization 900 may include an image (e.g., an RGB image, a depth image, an RGB-D image, etc.) of the target subject 148. The computing device 102 may identify or detect an obstructive sleep apnea event based on the data 125 (e.g., motion data, depth data) associated with the target subject 148.

For example, obstructive sleep apnea is associated with a blockage or obstruction of part or all of the upper airway of the target subject 148 during sleep. Example indicators of obstructive sleep apnea may include the diaphragm and chest muscles of the target subject 148 working relatively harder to open the upper airway and pull air into the lungs. Breathing by the target subject 148 may be very shallow, or the target subject 148 may even stop breathing for a temporal period. In some cases, the target subject 148 may start to breathe again with a loud gasp, snort, or body jerk. The computing device 102 may identify or detect an obstructive sleep apnea event based whether the data 125 (e.g., motion data, depth data) associated with the target subject 148 indicates one or more of the indicators (e.g., a degree of movement of the diaphragm and/or chest muscles exceeding a threshold amount, shallow breathing, etc.).

Aspects of the present disclosure described herein with respect to non-contact monitoring and depth sensing may include viewing patient data (e.g., historical, real-time, near real-time, etc.) in any environment, for example, via the computing device 102. Example environments include an operating room, an ICU, a NICU, a general care floor of a medical center, and a home environment, but are not limited thereto.

Aspects of the present disclosure support integrating image processing techniques (e.g., absolute minute volume, vent asynchrony detection, etc.), neonatal care monitoring techniques, and AI based techniques (e.g., touchless heart rate, respiratory failure prediction analytics, etc.) in generating breath visualization and respiratory waveforms as described herein. For example, the system 100 may support aggregation of data collected by non-contact monitoring devices (e.g., depth cameras, infrared cameras, thermal cameras, etc.). In some examples, the system 100 may support aggregation of data collected by using the techniques. For example, the system 100 may support aggregation of data collected using both non-contact monitoring devices (and techniques) and contact-based monitoring devices (and techniques).

As described herein, the system 100 may support areas such as respiratory rate, respiratory surveillance (e.g., PACU, PCA), hospital and home sleep monitoring, ventilators, and NICUs (e.g., respiratory rate, apnea, activity, caregiver interaction and stimulation, etc.). The system 100 may support continuous and accurate generation of breathing visualization and respiratory waveforms associated with a target subject 148, in any environment.

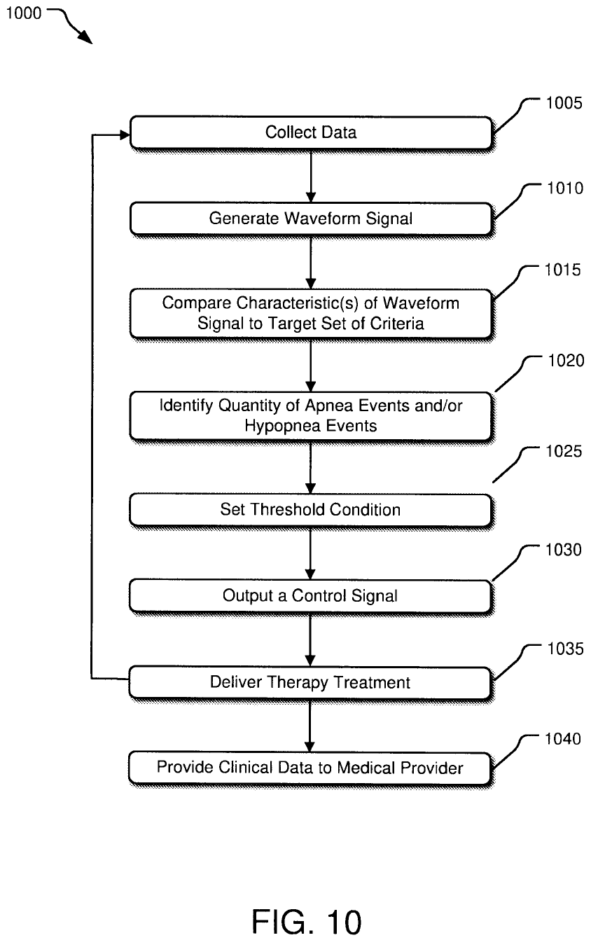

FIG. 10 illustrates an example of a process flow 1000 in accordance with aspects of the present disclosure. In some examples, process flow 1000 may implement aspects of the system 100 described herein.

In the following description of the process flow 1000, the operations may be performed in a different order than the order shown, or the operations may be performed in different orders or at different times. Certain operations may also be left out of the process flow 1000, or other operations may be added to the process flow 1000.

It is to be understood that any of the operations of process flow 1000 may be performed by any device (e.g., a computing device 102, another computing device 102, a server, etc.).

The process flow 1000 (and/or one or more operations thereof) may be carried out or otherwise performed, for example, by at least one processor. The at least one processor may be the same as or similar to the processor(s) 104 of the computing device 102 described above. A processor other than any processor described herein may also be used to execute the process flow 1000. The at least one processor may perform operations of the process flow 1000 by executing elements stored in a memory such as the memory 106. The elements stored in memory and executed by the processor may cause the processor to execute one or more operations of a function as shown in the process flow 1000. One or more portions of the process flow 1000 may be performed by the processor executing any of the contents of memory (e.g., monitoring engine 126, machine learning model(s) 138, processing algorithm(s) 142, etc.).

At 1005, the process flow 1000 includes collecting data (e.g., data 125) associated with a target subject (e.g., target subject 148). In an example, collecting the data includes receiving, from a depth sensing device (e.g., imaging device 124), motion data associated with the target subject. In some aspects, the motion data is free of image data. For example, the motion data may be substantially free of image data.

In some aspects, collecting the data includes receiving, from one or more sensing devices (e.g., imaging device 112), one or more biometric measurements (e.g., heart rate, temperature information, etc.) associated with the target subject.

At 1010, the process flow 1000 includes generating a waveform signal (e.g., waveform 155) indicative of the biometric parameter of the target subject based on the motion data associated with the target subject. In some aspects, the biometric parameter may include a respiration rate or a tidal volume.

In some cases, the process flow 1000 includes calculating one or more values of the biometric parameter based on the motion data.

In some cases, the process flow 1000 includes providing at least a portion of the motion data to a machine learning model (e.g., machine learning model 138). The process flow 1000 may include receiving an output from the machine learning model in response to the machine learning model processing at least the portion of the motion data, the output including one or more values of the biometric parameter.

Additionally, or alternatively, generating the waveform at 1010 may be based on the one or more biometric measurements.

At 1015, the process flow 1000 includes comparing the one or more characteristics of the waveform signal to a target set of criteria.

At 1020, the process flow 1000 includes identifying a quantity of apnea events, a quantity of hypopnea events, or both in association with a temporal duration, based on the waveform signal.

At 1025, the process flow 1000 includes setting a threshold condition based on one or more characteristics of the waveform signal.

In some aspects, the one or more characteristics of the waveform signal may include a pattern of the waveform signal.

In some aspects, the one or more characteristics of the waveform signal may include a ratio associated with a first amplitude (e.g., amplitude 160-a described with reference to FIG. 1) of the waveform signal and a second amplitude (e.g., amplitude 160-b described with reference to FIG. 1) of the waveform signal.

In some aspects, setting the threshold condition may include maintaining or modifying the threshold condition based on a result of the comparison at 1015. In some aspects, setting the threshold condition is based on the quantity of apnea events, the quantity of hypopnea events, or both in association with the temporal duration (e.g., as identified at 1020).

At 1030, the process flow 1000 includes outputting a control signal to a stimulation device (e.g., stimulation device 122) based on the waveform signal satisfying the threshold condition.

At 1035, the process flow 1000 includes delivering, via the stimulation device, a therapy treatment to the patient based on the control signal. In some cases, at 1035, the process flow 1000 includes outputting an alert based on the value of the biometric parameter, the one or more characteristics of the waveform signal, or both.

At 1040, the process flow 1000 includes providing clinical data to a medical provider. In some aspects, the clinical data may include a data record associated with the biometric parameter.

As noted above, the present disclosure encompasses methods with fewer than all of the steps identified in FIG. 10 (and the corresponding description of the process flow 1000), as well as methods that include additional steps beyond those identified in FIG. 10 (and the corresponding description of the process flow 1000). The present disclosure also encompasses methods that comprise one or more steps from one method described herein, and one or more steps from another method described herein. Any correlation described herein may be or comprise a registration or any other correlation.

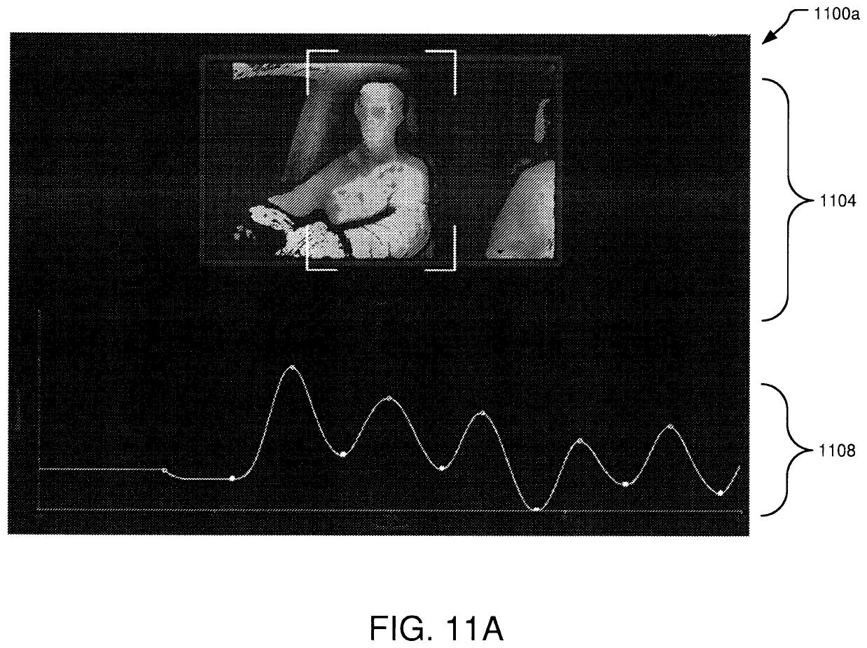

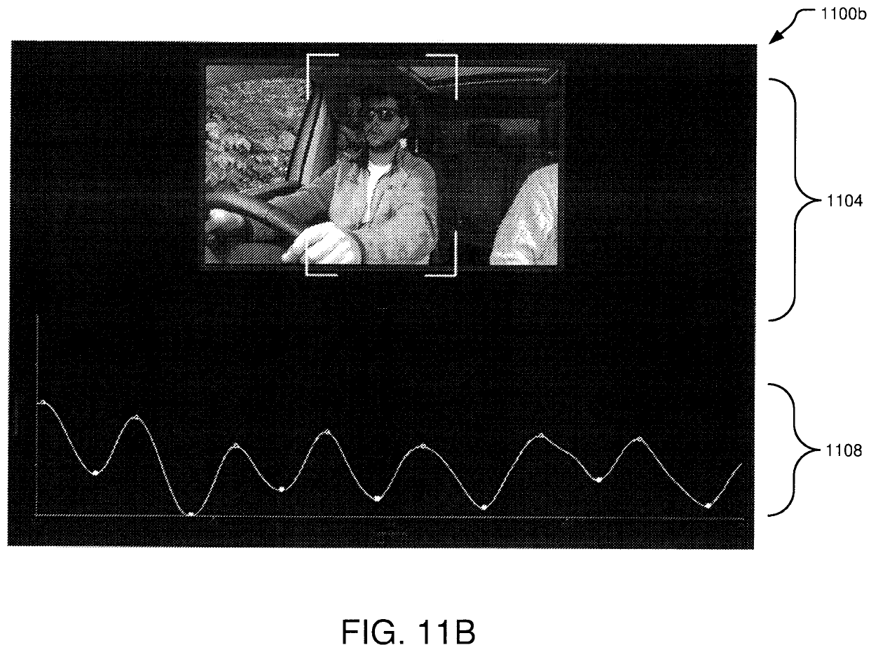

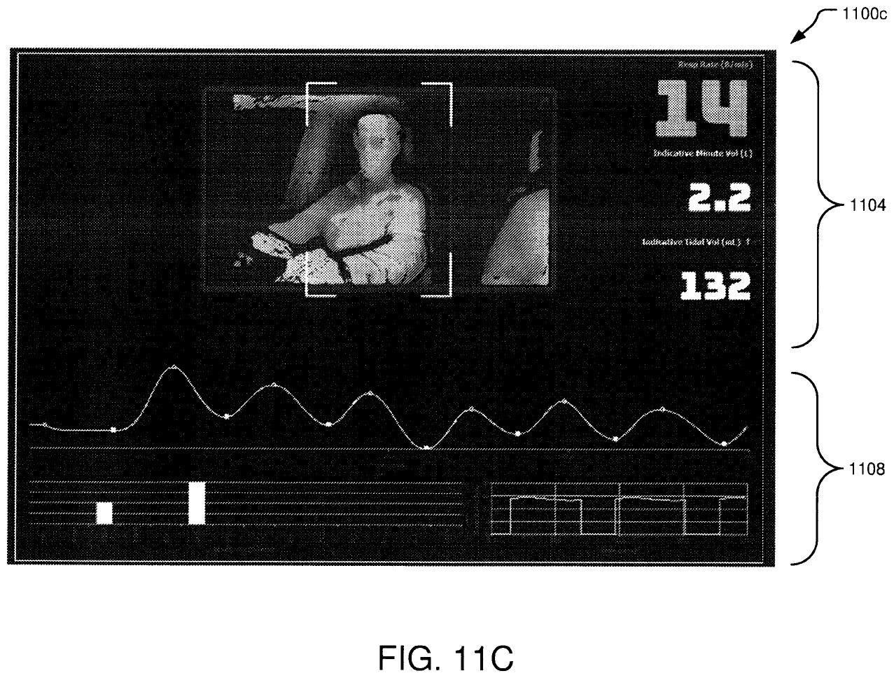

FIGS. 11A through 11C illustrates example visualizations 1100-a through 1100-c that support aspects of the present disclosure. In some aspects, the system 100 may support monitoring the target subject 148 (e.g., a driver) in a vehicle environment, in which the imaging device 124 is installed in the vehicle facing the target subject 148.

Each visualization 1100 (e.g., visualization 1100-a through visualization 1100-c) may include first information 1104 and second information 1108. First information 1104 may include an image (e.g., an RGB image, a depth image, an RGB-D image, etc.) of the target subject 148 and/or any additional information (e.g., respiratory information, volume information, tidal information, etc.) associated with the target subject 148. Second information 1108 may include a combination of waveforms, graphs, charts, etc. representative of the additional information. For example, the second information 1108 may include one or more graphs or waveforms indicating respiratory rate associated with the target subject 148. The computing device 102 may calculate the respiratory rate based on the data 125 (e.g., motion data, depth data) associated with the target subject 148.

In an example, referring to FIG. 11C, the first information 1104 indicates a respiratory rate of 14 breaths per minute, an indicative minute volume of 2.2 liters, and an indicative tidal volume of 132 milliliters.

The foregoing is not intended to limit the disclosure to the form or forms disclosed herein. In the foregoing Detailed Description, for example, various features of the disclosure are grouped together in one or more aspects, implementations, and/or configurations for the purpose of streamlining the disclosure. The features of the aspects, implementations, and/or configurations of the disclosure may be combined in alternate aspects, implementations, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, implementation, and/or configuration. Thus, the following claims are hereby incorporated into this Detailed Description, with each claim standing on its own as a separate preferred implementation of the disclosure.

Moreover, though the foregoing has included description of one or more aspects, implementations, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, implementations, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

It is to be appreciated that any feature described herein can be claimed in combination with any other feature(s) as described herein, regardless of whether the features come from the same described implementation.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an implementation that is entirely hardware, an implementation that is entirely software (including firmware, resident software, micro-code, etc.) or an implementation combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A system comprising:
a processor; and
a memory storing instructions thereon that, when executed by the processor, cause the processor to:
generate a waveform signal indicative of a biometric parameter of a target subject based at least in part on motion data associated with the target subject;
set a threshold condition based at least in part on one or more characteristics of the waveform signal; and
output a control signal to a stimulation device based at least in part on the waveform signal satisfying the threshold condition.

2. The system of claim 1, wherein the instructions are further executable by the processor to:
collect data associated with the target subject, wherein collecting the data comprises:
receiving, from a depth sensing device, the motion data associated with the target subject.

3. The system of claim 2, wherein collecting the data comprises:
receiving, from one or more sensing devices, one or more biometric measurements associated with the target subject,
wherein generating the waveform signal is based at least in part on the one or more biometric measurements.

4. The system of claim 1, wherein the instructions are further executable by the processor to:
compare the one or more characteristics of the waveform signal to a target set of criteria,
wherein setting the threshold condition comprises maintaining or modifying the threshold condition based at least in part on a result of the comparison.

5. The system of claim 1, wherein the one or more characteristics of the waveform signal comprise a pattern of the waveform signal.

6. The system of claim 1, wherein the one or more characteristics of the waveform signal comprise a ratio associated with a first amplitude of the waveform signal and a second amplitude of the waveform signal.

7. The system of claim 1, wherein the instructions are further executable by the processor to:
identify a quantity of apnea events, a quantity of hypopnea events, or both in association with a temporal duration, based at least in part on the waveform signal,
wherein setting the threshold condition is based on the quantity of apnea events, the quantity of hypopnea events, or both in association with the temporal duration.

8. The system of claim 1, wherein the instructions are further executable by the processor to:
deliver, via the stimulation device, a therapy treatment to the patient based at least in part on the control signal.

9. The system of claim 1, wherein the instructions are further executable by the processor to:
provide clinical data to a medical provider, wherein the clinical data comprises a data record associated with the biometric parameter.

10. The system of claim 1, wherein the instructions are further executable by the processor to:
output an alert based at least in part on the value of the biometric parameter, the one or more characteristics of the waveform signal, or both.

11. The system of claim 1, wherein the instructions are further executable by the processor to:
calculate one or more values of the biometric parameter based at least in part on the motion data.

12. The system of claim 1, wherein the instructions are further executable by the processor to:
provide at least a portion of the motion data to a machine learning model; and
receive an output from the machine learning model in response to the machine learning model processing at least the portion of the motion data, the output comprising one or more values of the biometric parameter.

13. A method comprising:
generating a waveform signal indicative of a biometric parameter of a target subject based at least in part on motion data associated with the target subject;
setting a threshold condition based at least in part on one or more characteristics of the waveform signal; and
outputting a control signal to a stimulation device based at least in part on the waveform signal satisfying the threshold condition.

14. The method of claim 13, further comprising:
collecting data associated with the target subject, wherein collecting the data comprises:
receiving, from a depth sensing device, the motion data associated with the target subject.

15. The method of claim 14, wherein collecting the data comprises:
comparing the one or more characteristics of the waveform signal to a target set of criteria,
wherein setting the threshold condition comprises maintaining or modifying the threshold condition based at least in part on result of the comparison.
